(19) United States
(12) Patent Application Publication
(10) Pub. No.: US 2006/0024658 A1
(43) Pub. Date: Feb. 2, 2006

(54) Composition and methods for evaluating an organism's response to alcohol or stimulants

(75) Inventors: Michael F. Miles, Mill Valley, CA (US); Chao-Qiang Lai, Belmont, MA (US); David J. Lockhart, Del Mar, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/746,794

(22) Filed: Dec. 23, 2003

Related U.S. Application Data
(63) Continuation of application No. 09/337,022, filed on Jun. 21, 1999, now abandoned.
(60) Provisional application No. 60/090,268, filed on Jun. 22, 1998.

(51) Int. Cl. C12Q 1/00; C12Q 1/68

(52) U.S. Cl. 435/4; 435/6

(57) ABSTRACT

This invention pertains to the identification of genes whose expression levels are altered by chronic exposure of a cell, tissue, or organism to one or more drugs of abuse (e.g. alcohol, stimulants, opiates, etc.). In one embodiment, this invention provides a method of monitoring the response of a cell a drug of abuse. The method involves contacting the cell with the drug of abuse; providing a biological sample comprising the cell; and detecting, in the sample, the expression of one or more genes or ESTs identified herein, where a difference between the expression of one or more of said genes or ESTs in said sample and one or more of said genes or ESTs in a biological sample not contacted with said drug of abuse indicates a response of the cell to the drug of abuse

### CROSS-REFERENCE TO RELATED APPLICATIONS

This application claims benefit under 35 U.S.C. §119(e) of provisional application U.S. Ser. No. 60/090,268, filed on Jun. 22, 1998, which is herein incorporated by reference in its entirety for all purposes.

### STATEMENT AS TO RIGHTS TO INVENTIONS MADE UNDER FEDERALLY SPONSORED RESEARCH AND DEVELOPMENT

Not Applicable

### FIELD OF THE INVENTION

This invention relates to the field of functional genomics. In particular this invention pertains to the identification of genes whose expression levels are altered by chronic exposure of a cell, tissue, or organism to one or more drugs of abuse.

### BACKGROUND OF THE INVENTION

Adaptive changes in central nervous system (CNS) function generate tolerance to and dependence on a used substances (e.g. drugs of abuse such as opiates, stimulants, and alcohol) as well as the craving which underlies addiction. There is theoretical and experimental evidence suggesting that changes in gene expression underlie central nervous system response consequent to chronic drug or alcohol expression. Yet particular alterations in gene regulation associated with CNS plasticity accompanying chronic drug abuse are unknown.

Substance abuse is a major public health problem in the United States and worldwide. For example, in this country alone it is estimated that alcoholism and alcohol abuse account for over 120 billion dollars in cost to society with lost productivity and medical costs secondary to ethanol-induced disease. Alcoholics suffer from a variety of end-organ diseases including liver cirrhosis, cardiac and skeletal myopathy, immune system dysfunction, peripheral neuropathy, and a number of degenerative diseases affecting the central nervous system. At the root of such “toxic” effects of alcohol lie several direct effects of ethanol in the central nervous system: namely, tolerance, dependence, and addiction.

On-going efforts have been focused on understanding the physiological role of several identified ethanol-responsive genes, as well as characterizing the mechanisms whereby ethanol regulated gene transcription. However, in order to more fully understand how changes in gene expression may contribute to the overall behavioral responses of an organism, there is a need to more fully catalogue the repertoire of ethanol-responsive genes in both cell culture and animal models. Such information will help elucidate the mechanisms underlying adaptive CNS changes occurring with chronic ethanol exposure. This could lead to new therapeutic interventions for treating alcoholism and alcohol-related neurological disease. Furthermore, the identification of ethanol-responsive genes will also provide candidate genes for application in genetic studies on alcoholism.

### SUMMARY OF THE INVENTION

This invention this invention pertains to the identification of genes whose expression levels are altered by chronic or acute exposure of a cell, tissue, or organism to one or more drugs of abuse (e.g. stimulants, opiates, alcohol, nicotine, etc.). Having identified genes (or ESTs) whose regulation is altered when the organism is subjected to one or more drugs of abuse, the expression of these genes can be utilized in a wide variety of assays. Thus, for example, the expression levels of one or more of these genes can be used for evaluating drug treatments, for identifying susceptibility to alcoholism and/or drug dependency, and for assaying the response of an organism to a drug or to an agent believed to modulate the response of an organism to a drug. The genes also provide a useful starting point for locating polymorphisms relating to alcohol/drug abuse/dependency. The genes/ESTs also provide good targets for screening for drugs that alter the response of an organism to one or more drugs of abuse.

Thus, in one embodiment, this invention provides methods of monitoring the response of a cell to a drug of abuse. The methods involve contacting the cell with the drug of abuse; providing a biological sample comprising the cell; and detecting, in the sample, the expression of one or more genes or ESTs selected from the group consisting of the genes and ESTs of Table 1, the genes and ESTs of Table 2, the genes and ESTs of Table 3 the genes and ESTs of Table 4, the genes and ESTs of Table 5, and the genes and ESTs of Table 6, where a difference between the expression of one or more of said genes or ESTs in said sample and one or more of said genes or ESTs in a biological sample not contacted with said drug of abuse indicates a response of said cell to the drug of abuse.

In particularly preferred embodiments, the just the expression of genes of any one or more of Tables 1-6 is assayed, while in other preferred embodiments, just the expression of ESTs of any one or more of Tables 1-6 is assayed. In particularly preferred methods the genes or ESTs are selected from the group consisting of dopamine β-hydroxylase (DBH), sodium-dependent norepinephrine transporter (NET), delta-like protein (DLK), and monocyte chemoattractant peptide 1 (MCP-1).

The drug of abuse can include an alcohol, a stimulant, and opiate, and the like. In some embodiments, the drug of abuse is selected from the group consisting of cocaine, amphetamine, methamphetamine, ephenedrine, methylphenidate, and methcathinone. In other embodiments, the contacting can involve contacting the contacting comprises contacting the cell (in culture, in a tissue (in culture or in an organism), in an organism, etc) with an alcohol (e.g. ethanol, propanol, methanol, etc.). 1. In one particularly preferred embodiment, the drug of abuse is ethanol or cocaine. Preferred test organisms include, but are not limited to a human, a non-human primate, a rodent, a porcine, a lagomorph, a canine, a feline, and a bovine.

The detecting can involve detecting a protein fully or partially, encoded by one of the genes or ESTs identified herein. Thus, for example, the protein can be detected via capillary electrophoresis, a Western blot, mass spectroscopy, immunochromatography, or immunohistochemistry. In another embodiment, the detecting can involve obtaining a nucleic acid from the cell and hybridizing said nucleic acid to one or more probes that specifically hybridize to said genes or ESTs under stringent conditions. The hybridization can be by any of a variety of methods including, but not limited to a Northern blot, a Southern blot, an array hybridization, an affinity chromatography, and an in situ hybridization. In some particularly preferred methods the one or more probes is a plurality of probes that forms an array of probes. Such arrays include arrays of probes comprising at least about 1000 different probes and/or having a probe density of at least about 1000 different probes per cm². The probes, in some embodiments, are chemically synthesized oligonucleotides covalently linked to a solid support, while in other embodiments, the probes are spotted onto a solid support. The array can include includes one or more probes that specifically hybridize to a housekeeping gene (e.g., an actin gene, a G6PDH gene, etc).

In another embodiment, this invention provides methods of screening for an agent that alters the response of a cell to a drug of abuse. In preferred embodiments, the methods are essentially the same as the methods of monitoring the response of a cell to a drug of abuse except that the cell is also contacted with the agent that is being screened for activity. In this case, a difference in the expression level of one or more of the genes or ESTs in the sample, as compared to the genes or ESTs in a sample not contacted with the test agent indicates that the test agent alters the response of said cell to the drug of abuse.

In still another embodiment, this invention provides nucleic acid arrays for monitoring the response of a cell to a drug of abuse (e.g. alcohol, stimulant, opioid, etc.). In a preferred embodiment, the array comprises a plurality of nucleic acid probes attached to a solid support. Preferred arrays predominantly contain nucleic acid probes that hybridize under stringent conditions to nucleic acids selected from the group consisting of the genes and ESTs of Table 1, the genes and ESTs of Table 2, the genes and ESTs of Table 3 the genes and ESTs of Table 4 the genes and ESTs of Table 5, and the genes and ESTs of Table 6. Preferred arrays include (sometimes predominate in) probes that hybridize under stringent conditions to one or more nucleic acids that hybridize specifically to a nucleic acid selected from the group consisting of dopamine β-hydroxylase (DBH), sodium-dependent norepinephrine transporter (NET), delta-like protein (DLK), and monocyte chemoattractant peptide 1 (MCP-1). Preferred arrays have the probe number and/or densities described herein and include chemically synthesized and/or spotted arrays.

In still another embodiment, this invention provides methods of making a nucleic acid probe array for monitoring the response of a cell to a drug of abuse (e.g. alcohol, a stimulant, an opioid, etc.). The methods involve attaching to a surface, one or more nucleic acid probes that specifically hybridize to a nucleic acid selected from the group consisting of the genes and ESTs of Table 1, the genes and ESTs of Table 2, the genes and ESTs of Table 3 the genes and ESTs of Table 4 the genes and ESTs of Table 5, and the genes and ESTs of Table 6. The methods can fabricating the arrays so that they predominantly contain the probes identified herein. In particularly preferred methods, nucleic acids include probes that hybridize under stringent conditions to a nucleic acid selected from the group consisting of dopamine β-hydroxylase (DBH), sodium-dependent norepinephrine transporter (NET), delta-like protein (DLK), and monocyte chemoattractant peptide 1 (MCP-1). In one embodiment, the probes are chemically synthesized oligonucleotides covalently linked to a solid support, while in another embodiment, the probes are spotted onto a solid support. Preferred arrays are fabricated to have probe numbers and/or probe densities as described herein. The arrays can also include control probes specific to housekeeping genes and/or one or more mismatch control probes.

This invention also provides a nucleic acid construct comprising a nucleic acid probe selected from the group consisting of the genes and ESTs of Table 1, the genes and ESTs of Table 2, the genes and ESTs of Table 3 the genes and ESTs of Table 4 the genes and ESTs of Table 5, and the genes and ESTs of Table 6; an origin or replication; and a promoter. Also included are vector(s) comprising the nucleic acid construct, compositions the vector and a carrier, host cell(s) transfected the nucleic acid construct, and host cell(s) transfected with the vector.

Also provided are methods of amplifying a probe. These methods involve culturing the host cell (containing the vector and/or nucleic acid construct) in a growth medium and under amplifying conditions; and allowing the construct to accumulate. The methods can also further involve separating the construct from the medium and the cells.

In still another embodiment, this invention provides kits for practice of the methods of this invention. Preferred kits include a container containing one or more of the arrays described herein. Optionally included are any of the reagents, labels, probes, etc. described herein. Also optionally included are instructional materials describing the use of the arrays in one or more of the assays described herein.

Definitions

The term “immunoassay” is an assay that utilizes an antibody to specifically bind an analyte. The immunoassay is characterized by the use of specific binding properties of a particular antibody to isolate, target, and/or quantify the analyte.

The term “nucleic acid” refers to a deoxyribonucleotide or ribonucleotide polymer in either single- or double-stranded form, and unless otherwise limited, encompasses known analogs of natural nucleotides that can function in a similar manner as naturally occurring nucleotides.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The phrase “the genes or ESTs of Table X” refers to the genes or ESTs listed in Table X (e.g. one or Tables 1-6). The term refers to any of the nucleic acid sequences identified in the referenced table whether or not it is a gene or EST. In preferred embodiments the term also includes human homologues of the gene or EST where the listed gene or EST is non-human. In addition, the EST also is intended to include a gene of which the EST is a component.

A “nucleic acid probe” is defined as a nucleic acid capable of binding to a target nucleic acid of complementary sequence through one or more types of chemical bonds, usually through complementary base pairing, usually through hydrogen bond formation. As used herein, a probe may include natural (i.e. A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions.

The term “antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Fundamental Immunology, Third Edition, W. E. Paul, ed., Raven Press, N.Y. 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies, those synthesized de novo using recombinant DNA methodologies (e.g., single chain Fv), and those found in display libraries (e.g. phage display libraries).

The term “drugs of abuse” refers to drugs that are psychoactive and that induce tolerance and/or addiction. Drugs of abuse include, but are not limited to stimulants (e.g. cocaine, amphetamines), opiates (e.g. morphine, heroin), nicotine, alcohol, and the like. In addition, when referring to contacting a cell with a drug of abuse the term can include contacting the cell with a metabolic product of a drug of abuse (e.g. cotinine).

The phrases “hybridizing specifically to” or “specific hybridization” or “selectively hybridize to”, refer to the binding, duplexing, or hybridizing of a nucleic acid molecule preferentially to a particular nucleotide sequence under stringent conditions when that sequence is present in a complex mixture (e.g., total cellular) DNA or RNA.

The term “stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and northern hybridizations are sequence dependent, and are different under different environmental parameters. An extensive guide to the hybridization of nucleic acids is found in Tijssen (1993) Laboratory Techniques in Biochemistry and Molecular Biology—Hybridization with Nucleic Acid Probes part I chapter 2 Overview of principles of hybridization and the strategy of nucleic acid probe assays, Elsevier, N.Y. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe.

An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, Sambrook et al. (1989) Molecular Cloning—A Laboratory Manual (2nd ed.) Vol. 1-3, Cold Spring Harbor Laboratory, Cold Spring Harbor Press, NY, (Sambrook et al.) supra for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. In general, a signal to noise ratio of 2× (or higher) than that observed for an unrelated probe in the particular hybridization assay indicates detection of a specific hybridization. Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

In one particularly preferred embodiment, stringent conditions are characterized by hybridization in 1 M NaCl, 10 mM Tris-HCl, pH 8.0, 0.01% Triton X-100, 0.1 mg/ml fragmented herring sperm DNA with hybridization at 45° C. with rotation at 50 RPM followed by washing first in 0.9 M NaCl, 0.06 M NaH₂PO₄, 0.006 M EDTA, 0.01% Tween-20 at 45° C. for 1 hr, followed by 0.075 M NaCl, 0.005 M NaH₂PO₄, 0.5 mM EDTA at 45° C. for 15 minutes.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides, refers to two or more sequences or subsequences that have at least 60%, preferably 80%, most preferably 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

The term “biological sample” refers to sample is a sample of biological tissue, cells, or fluid that, in a healthy and/or pathological state, contains an a nucleic acid or polypeptide that is to be detected according to the assays described herein. Such samples include, but are not limited to, cultured cells, acute cell preparations, sputum, amniotic fluid, blood, blood cells (e.g., white cells), tissue or fine needle biopsy samples, urine, peritoneal fluid, and pleural fluid, or cells therefrom. Biological samples may also include sections of tissues such as frozen sections taken for histological purposes. Although the sample is typically taken from a human patient, the assays can be used to detect ESX genes or gene products in samples from any mammal, such as dogs, cats, sheep, cattle, and pigs, etc. The sample may be pretreated as necessary by dilution in an appropriate buffer solution or concentrated, if desired. Any of a number of standard aqueous buffer solutions, employing one of a variety of buffers, such as phosphate, Tris, or the like, at physiological pH can be used.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecules” refers to molecules of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

### BRIEF DESCRIPTION OF THE DRAWINGS

FIGS. 1A, 1B, and 1C illustrate induction of genes associated with cocaine sensitization. FIG. 1A shows the response of FAK, myogenin, GluR-2, and K+ch.sub in VTA. FIG. 1B shows the response of Icfa CoA-ligase, PS synthase, MAP2, and ARF5 in VTA. FIG. 1C shows the response of genes in the nucleus accumbens.

FIGS. 2A and 2B illustrate the results of an initial study of the effects of alcohol on gene expression. FIG. 2A illustrates the relationship between gene expression and ethanol dosage. FIG. 2B shows the effects of various alcohols on gene expression.

FIG. 3A shows the summary of final selected genes, and the magnitude of change in expression levels when the cells are treated with 100 mM ethanol, 72 hours. Genes are arranged into functional groups.

FIG. 3B shows the dose response results for the four major response genes identified herein.

FIGS. 4A, 4B, and 4C show the effect of ethanol on expression levels of DBH, DLK, NET, MCP, and GPD. FIG. 4A shows Northern blot data from SHSY cells. FIG. 4B shows Western blot data for DBH from cells exposed to 150 mM ethanol for 72 h. FIG. 4C shows ELIZA data for MCP-1.

FIG. 5 shows RT-PCR data for DBH in adrenal gland of control vs. ethanol treated mice.

### DETAILED DESCRIPTION

This invention pertains to the discovery of a number of genes whose expression levels are altered upon chronic exposure to substances of abuse (e.g. opiates, stimulants (e.g., cocaine), alcohol, etc.). Identification of such genes provides information regarding the molecular events underlying central nervous system changes accompanying tolerance and addiction, provides unique targets to screen for agents that will modulate the central nervous system response to drugs of abuse, and provides assays to evaluate the effect of such agents on cells, tissues, or organisms.

Exposure of laboratory animals or human volunteers to repeated doses of ethanol will induce tolerance. This is characterized by the animal or human requiring higher blood/brain levels of ethanol to produce the same intoxicating action seen in a naive individual. Alcoholics can achieve a remarkable level of tolerance such that they can appear sober at brain ethanol levels that would kill a normal individual. This is not due to increased metabolism of the drug but rather represents a fundamental plasticity of the nervous system such that relatively normal CNS functioning can occur at very high ethanol levels. This adaptation produces a deleterious response, however, in that the organism is now dependent up on ethanol for normal CNS functioning. Withdrawal from ethanol at this point would be accompanied by sympathetic hyperactivity, seizures, hallucinations and, in a significant number of cases, death due to circulatory collapse.

Studies in both humans and animals have shown that tolerance can be generated within a relatively short period of time, that is, within 48-72 hours of initiating a steady intake of ethanol. The duration of the withdrawal soquelae accompanying dependence follows a similar time course.

Addiction to drugs, in contrast to tolerance and dependence, involves an increased desire to seek the drug. A variety of data suggests that early and late adaptive changes in gene expression in brain areas subserving reward centers may lead to the plasticity that generates addiction. See, for example, Nestler et al. (1993) Neuron 11: 995-1006. Sensitization to the locomotor activating effects of abused drugs has been widely used as a model for studying events leading to addiction (see, e.g., Phillips et al. (1997) Crit. Rev. Neurobiol., 11: 21-33). Animals will exhibit increasing locomotor activity following repeated exposure to drugs of abuse—hence sensitization. For example, exposure or treatment of a naive animal with cocaine will cause an increase in locomotor activity that can be quantitated using a computerized photo-beam crossing square. Subsequent doses of cocaine, administered once a day, will cause a progressive increase in this locomotor activation response. Similar sensitization will occur with exposure to amphetamines, opiates, nicotine, and ethanol. Remarkably, sensitization to a drug can persist for many weeks or months of drug abstinence. Sensitization can therefore be used as a model to study CNS plasticity in drug addiction. Changes in gene expression accompanying sensitization may well be related to the molecular events involving the establishment of drug craving behaviors.

I. Genes and ESTs Associated with

A) Uses of Genes and ESTs Whose Expression is Altered by Drugs of Abuse.

This invention pertains to the identification of a number of genes and ESTs whose expression is altered by chronic exposure of a cell, tissue or organism to one or more drugs of abuse (e.g. alcohol, cocaine, opiates, etc.). The identification of genes whose regulation is altered in alcohol tolerance and/or addiction provides a valuable tool to evaluate the response of a cell, tissue, or organism to one or more drugs of abuse. Evaluation of the nature of the response provide information useful in designing therapeutic, e.g. recovery, regimen, in evaluating the susceptibility of the organism or patient to drugs of abuse (e.g. opiates) in a medical context, and in characterizing an organisms response to a drug of abuse or a therapeutic drug used in the treatment of addiction.

Monitoring expression of the genes and/or ESTs identified herein also provides a mechanism by which test agents can be screened for the ability to alter (modulate) the response of a cell, tissue, or organism to one or more drugs of abuse.

Thus, in one embodiment, this invention provides methods of monitoring the response of a cell (e.g. a cell in culture, in tissue, in an organism, etc.) to one or more drugs of abuse. Generally such methods involve contacting the cell with one or more drugs of abuse (or their metabolic by-products), providing a biological sample comprising the cell and detecting the expression level(s) in the sample of one or more genes and/or ESTs listed in Tables 1-6 (optionally excluding the α7 subunit of the neuronal acetylcholine receptor (nAChRα7)). As explained herein, the detection can involve detection of a change in gene copy number and/or a change in transcribed mRNA level(s) and/or a change in translated protein, and/or a change in protein activity. Typically the change will be monitored relative to control cell(s) that have not been contacted with the drug(s) of abuse.

In another embodiment this invention provides methods of screening test agents for the ability to alter a cell's, tissues, or organism's response to a drug of abuse. This involves contacting a cell to the test agent either in the presence of the drug of abuse, or after exposure (e.g. chronic exposure) of the cell to the drug of abuse, providing a biological sample comprising the cell and detecting the expression level(s) in the sample of one or more genes and/or ESTs listed in Tables 1-6 (optionally excluding the α7 subunit of the neuronal acetylcholine receptor (nAChRα7)). Those test agents that alter the expression levels of one or more of the genes and/or ESTs in Tables 1-6 provide good therapeutic lead compounds.

It is also possible to screen test agents for the ability to modulate the cell's response to a drug of abuse by screening for binding of that agent to the gene, mRNA or translated protein of the genes or ESTs of Tables 1-6 (including human homologues of the mouse genes or ESTs). Binding assays are well know to those of skill in the art.

Having identified genes and/or ESTs involved in the response of a cell, tissue, or organism to exposure to a drug of abuse, this information can be used to design modulators of such a response or to elucidate the mechanisms of such a response. Thus, for example, the activity of one or more of the genes and/or ESTs identified in Tables 1-6 can be elucidated by “knocking out” the gene or EST with the use of antisense molecules (e.g. antisense nucleic acids), the use of gene/mRNA-specific ribozymes, or by production of knockout animals (e.g. knockout mice) where in which the gene(s) of interest are disrupted so that they do not produce the normal gene product.

B) Genes and ESTs Whose Regulation is Altered by Drugs of Abuse.

Genes and ESTs whose expression is altered by contact of a cell with a drug of abuse (e.g. alcohol or cocaine) were identified by exposing human neuroblastoma cells (SH-SY5Y-AH1861 cell line). For gene expression analysis, cells were treated for 72 h in the absence or presence of 50, 100 or 150 mM ethanol.

In addition, animal studies were conducted on female DBA/2J mice (Simonsen Laboratories, Gilroy, Calif.) weighing 20-30 g at 8 weeks of age. The animals were injected intraperitoneally with 4 g/kg ethanol or saline at 10:00 am, returned to their home cage, and killed 6 or 24 h later and the tissues analyzed for alterations in gene expression levels.

The gene expression levels were monitored using Affymetrix GeneChip Hu6800 set including 4 probe arrays (A, B, C, D) of over 65,000 different oligonucleotides each. Oligonucleotides were complementary to 5,800 full-length human cDNA based on sequence information from the UniGene, GenBank and TIGR databases. Each gene was represented by an average of 20 different pairs of 20-25 mer oligonucleotides.

Preferred genes and ESTs whose expression was altered by exposure to ethanol are identified in Table 1. In particular, four genes showed a dose-dependent manner response to ethanol and are therefore believe to represent important targets of ethanol. These genes are DBH (dopamine β hydroxylase) an enzyme catalyzing the formation of norepinephrine (NE), NET (sodium-dependent NE transporter), DLK (delta-like protein), and MCP-1 (monocyte chemoattractant peptide 1). Gene CHRNA7, a nAChR alpha 7 subunit has previously been shown to be regulated by ethanol and, in certain preferred embodiments, is excluded from the assays of this invention.

TABLE 1
Most preferred genes/ESTs whose expression is altered by exposure to ethanol.

| Gene ID | E100 | Provisional Functional Class | Acc# | Gene Name |
|---|---|---|---|---|
| PGY1 | 2.3 | cell defense/homeostasis | M29447 | P glycoprotein 1/multiple drug |
| | | | | resistance 1 |
| GSTM4 | 0.9 | cell defense/homeostasis | M99422 | Glutathione S-transferase M4 |
| E2-28.4 (EST) | 1 | cell defense/homeostasis | R01227 | ESTs, Highly similar to |
| | | | | UBIQUITIN-CONJUGATING |
| | | | | ENZYME E2-28.4 KD |
| NAIP | 0.9 | cell defense/homeostasis | U19251 | Neuronal apoptosis inhibitory |
| | | | | protein |
| GLRX | 1.4 | cell defense/homeostasis | X76648 | Glutaredoxin (thioltransferase) |
| RAC2 | −0.9 | cytoskeleton protein and regulator | H42477 | Ras-related C3 botulinum toxin |
| | | | | substrate 2 (rho family, small |
| | | | | GTP binding protein Rac2) |
| ARHGDIB | −0.7 | cytoskeleton protein and regulator | L20688 | RHO GDP-DISSOCIATION |
| | | | | INHIBITOR 2 |
| SSH3BP1 | 0.7 | cytoskeleton protein and regulator | R34245 | Spectrin SH3 domain binding |
| | | | | protein 1 (?Verprolin) |
| KRT18 | −2.3 | cytoskeleton protein and regulator | T53412 | Keratin type I cytoskeleton 18 |
| NEF3 | 1.4 | cytoskeleton protein and regulator | Y00067 | NFM |
| MGP | −0.7 | extracellular matrix protein | H52207 | Matrix Gla protein |
| SPARC | −1.3 | extracellular matrix protein | T54767 | SPARC |
| LUM | 1.6 | extracellular matrix protein | U21128 | Lumican |
| NP | 0.5 | metabolism | T47964 | Purine nucleoside phosphorylase |
| GCH1 | 1.6 | metabolism | U19523 | GTP Cyclohydrolase |
| DBH | 5.4 | metabolism | X13255 | Dopamine beta-hydroxylase |
| GPI-H | −0.6 | protein synthesis/proc. | L19783 | GPI-H |
| RPL14 | −0.5 | protein synthesis/proc. | R82938 | Ribosomal protein L14 |
| CPE | 1.2 | protein synthesis/proc. | X51405 | Carboxypeptidase E |
| EGFR | 1 | signaling molecule | H02836 | EGF receptor |
| HIRH | −1 | signaling molecule | H14506 | Pre-B cell growth stimulating |
| | | | | factor |
| IL7 | −0.7 | signaling molecule | J04156 | IL7 |
| MCP1 | −1.9 | signaling molecule | M26683 | MCP-1 (interferon gamma |
| | | | | inducible mRNA) |
| SLC6A2(NET) | 2.6 | signaling molecule | M65105 | NET |
| NSMAF | 1.1 | signaling molecule | R41765 | FAN protein (Hypothetical Trp- |
| | | | | Asp repeats containing prot) |
| DLK1 | 2.9 | signaling molecule | T49117 | dlk |
| TMPO | 1 | signaling molecule | U09086 | Thymopoietin |
| DUSP4 | 0.9 | signaling molecule | U21108 | Dual specific phosphatase |
| NPTX2 | 0.9 | signaling molecule | U29195 | NPTX2 |
| NFIB2/3 | 2.1 | transcription factor | H91713 | NFI-B3 (CCAAT box-binding |
| | | | | TF) |
| FKHL1 | −0.9 | transcription factor | R60332 | Trancription factor BF1 |
| ZNF42 | −1.9 | transcription factor | R83364 | Zinc finger protein 42 |
| TP53 | −1.1 | transcription factor | X54156 | p53 |
| PRHX | 1 | transcription factor | X67235 | Proline rich homeobox |
| SOX9 | −1.2 | transcription factor | Z46629 | SOX9 |
| EST | 0.9 | Unknown | H08637 | (NF1) |
| PMSCL2 | 0.7 | Unknown | R40490 | Autoantigen PM-SCL |
| EST | −1.3 | Unknown | R47985 | (Acrosin) |
| EST | −0.8 | Unknown | R60751 | (IEP2) |
| EST | −3.7 | Unknown | R73461 | (TCRbeta) |
| EST | 1 | Unknown | T94087 | (JNK2) |

Earlier studies of the effects of the effects of cocaine on gene expression in mice are shown in Tables 2-5. In these studies, mice were sensitized to cocaine by repeated administration. Sensitization refers to an increase in locomotor activity that occurs following repeated exposure to drugs of abuse. Sensitization is stable for long periods of drug abstinence and thus clearly represents a plasticity that generates an increased CNS response to abused drugs—as seen with addiction.

The mice used in these studies were treated with intra peritoneal injection of cocaine (10 mg.kg) or saline every other day for up to 12 days. Behavioral testing for locomoter activity was done on each injection day. Acute treatment was a single dose of cocaine.

Table 2 identifies genes and/or ESTs whose expression is altered by cocaine sensitization as assayed in mouse hippocampus. Similarly, Tables 3, 4, and 4 identify genes and/or ESTs whose expression is altered by cocaine sensitization as assayed in ventral tegmental area, prefrontal cortex, and nucleus accumbens respectively.

TABLE 2
Altered gene expression in mouse hippocampus due to cocaine sensitization..

| Gene Name | Accession # | Gene ID |
|---|---|---|
| Msa.30464.0 | AA097203 | Homologous to sp P25439: HOMEOTIC GENE REGULATOR |
| | | (BRAHMA PROTEIN). |
| Msa.4409.0 | AA138226 | Homologous to sp P09497: CLATHRIN LIGHT CHAIN B (BRAIN |
| | | AND LYMPHOCYTE LCB). |
| Msa.972.0 | Y00305 | Mouse MBK1 mRNA for mouse brain potassium channel protein-1 |
| Msa.26665.0 | AA064355 | Homologous to sp P18266: GLYCOGEN SYNTHASE KINASE-3 |
| | | BETA (EC 2.7.1.37) (GSK-3 BETA) (FACTOR A) (FA). |
| Msa.13420.0 | W57194 | Homologous to sp P34547: PROBABLE UBIQUITIN CARBOXYL- |
| | | TERMINAL HYDROLASE R10E11.3 (EC 3.1.2.15) (UBIQUITIN |
| | | THIOLESTERASE) (UBIQUITIN-SPECIFIC PROCESSING |
| | | PROTEASE) (DEUBIQUITINATING ENZYME). |
| Msa.18914.0 | AA007816 | Homologous to sp P25439: HOMEOTIC GENE REGULATOR |
| | | (BRAHMA P |
| Msa.22537.0 | AA035915 | Homologous to sp P17082: RAS-LIKE PROTEIN TC21 |
| | | (TERATOCARCINOMA ONCOGENE). |
| Msa.1293.0 | L04961 | Mouse Xist (X inactive specific transcript) mRNA for open reading |
| | | frame |
| Msa.18213.0 | AA000227 | Homologous to sp Q09103: EYE-SPECIFIC DIACYLGLYCEROL |
| | | KINASE (EC 2.7.1.107) (RETINAL DEGENERATION A PROTEIN) |
| | | (DIGLYCERIDE KINASE) (DGK). |
| Msa.14403.0 | W65084 | Homologous to sp P41220: G0/G1 SWITCH REGULATORY |
| | | PROTEIN 8. |
| Msa.3122.0 | U41736 | M. musculus ancient ubiquitous 46 kDa protein AUP1 precursor (Aup1) |
| | | mRNA, complete cds |
| Msa.22541.0 | AA035984 | Homologous to sp P23246: MYOBLAST CELL SURFACE ANTIGEN |
| | | 24.1D5 (FRAGMENT). |
| Msa.2652.0 | X83933 | Mouse RyR2 mRNA for cardiac ryanodine receptor, partial cds |
| Msa.7305.0 | W18385 | Homologous to sp P20340: RAS-RELATED PROTEIN RAB-6. |
| Msa.3904.0 | AA153265 | Homologous to sp Q01485: ANKYRIN, BRAIN VARIANT 2 |
| | | (ANKYRIN B) (ANKYRIN, NONERYTHROID) (FRAGMENT). |
| Msa.7689.0 | W20652 | Homologous to sp P35214: 14-3-3 PROTEIN GAMMA (PROTEIN |
| | | KINASE C INHIBITOR PROTEIN-1) (KCIP-1). |
| Msa.2405.0 | X70764 | M. musculus mRNA for serine/threonine protein kinase |
| Msa.32377.0 | AA107999 | Homologous to sp P45890: ACTIN-LIKE PROTEIN 13E. |
| Msa.34345.0 | AA117492 | Homologous to sp P36887: CAMP-DEPENDENT PROTEIN KINASE, |
| | | ALPHA-CATALYTIC SUBUNIT (EC 2.7.1.37) (PKA C-ALPHA) |
| | | (FRAGMENT). |
| Msa.3187.0 | U69270 | M. musculus LIM domain binding protein 1 (Ldb1) mRNA, complete |
| | | cds |
| Msa.40717.0 | AA155191 | Homologous to sp P33176: KINESIN HEAVY CHAIN. |
| Msa.2788.0 | U56649 | M. musculus cyclic nucleotide phosphodiesterase (PDE1A2) mRNA, |
| | | complete cds |
| Msa.868.0 | J03236 | M. musculus transcription factor junB (junB) gene, 5′ region and |
| | | complete cds |
| Msa.37527.0 | AA138791 | Homologous to sp P20936: GTPASE-ACTIVATING PROTEIN (GAP) |
| | | (RAS P21 PROTEIN ACTIVATOR). |
| Msa.3063.0 | D87903 | Mouse mRNA for ARF6, complete cds |
| Msa.10386.0 | AA125097 | Homologous to sp P10495: GLYCINE-RICH CELL WALL |
| | | STRUCTURAL PROTEIN 1 |
| Msa.3189.0 | U75321 | M. musculus chromaffin granule ATPase II homolog mRNA, complete |
| | | cds |
| Msa.21971.0 | AA154451 | Homologous to sp P27694: REPLICATION PROTEIN A 70 KD DNA- |
| | | BINDING SUBUNIT (RP-A) (RF-A) (REPLICATION FACTOR-A |
| | | PROTEIN 1) (SINGLE-STRANDED NA-BINDING PROTEIN). |
| Msa.35530.0 | AA119959 | Homologous to sp P15303: PROTEIN TRANSPORT PROTEIN |
| | | SEC23. |
| Msa.9908.0 | W42216 | Homologous to sp P25439: HOMEOTIC GENE REGULATOR |
| | | (BRAHMA PROTEIN). |
| Msa.29918.0 | AA087943 | Homologous to sp P12714: ACTIN, CYTOPLASMIC BETA. |
| Msa.3283.0 | U51037 | M. musculus 11-zinc-finger transcription factor (CTCF) mRNA, |
| | | complete cds |
| Msa.2629.0 | X84239 | M. musculus mRNA for rab5b protein |
| Msa.21307.0 | AA023589 | Homologous to sp P30725: DNAJ PROTEIN. |
| Msa.11233.0 | W50127 | Homologous to sp P06687: SODIUM/POTASSIUM- |
| | | TRANSPORTING ATPASE ALPHA-3 CHAIN (EC 3.6.1.37) |
| | | (SODIUM PUMP) (NA+/K+ ATPASE) (ALPHA(III)). |
| Msa.3242.0 | D50263 | Human mRNA for unknown product, complete cds |
| Msa.10796.0 | W49135 | Homologous to sp P00848: ATP SYNTHASE A CHAIN (EC 3.6.1.34) |
| | | (PROTEIN 6). |
| Msa.2075.0 | U58471 | House mouse; M. domesticus day 14 embryo whole embryo mRNA for |
| | | NeuroD-related factor (NDRF) containing a bHLH domain, complete |
| | | cds |
| Msa.596.0 | X76654 | M. musculus ear-2 transcription factor mRNA, complete cds |
| Msa.2463.0 | X63440 | M. musculus mRNA for P19-protein tyrosine phosphatase |
| Msa.29072.0 | AA073600 | Homologous to sp Q01485: ANKYRIN, BRAIN VARIANT 2 |
| | | (ANKYRIN B) (ANKYRIN, NONERYTHROID) (FRAGMENT). |
| Msa.40752.0 | AA155148 | Homologous to sp P17097: ZINC FINGER PROTEIN 7 (ZINC |
| | | FINGER PROTEIN KOX4) (ZINC FINGER PROTEIN HF.16). |
| Msa.2088.0 | X01023 | Mouse normal c-myc gene and translocated homologue from J558 |
| | | plasmocytoma cells (cDNA sequence) |
| Msa.8882.0 | W34756 | Homologous to sp P31218: URIDINE KINASE (EC 2.7.1.48) |
| | | (URIDINE MONOPHOSPHOKINASE) (PYRIMIDINE |
| | | RIBONUCLEOSIDE KINASE). |
| Msa.39606.0 | AA146282 | Homologous to sp P15092: INTERFERON-ACTIVATABLE |
| | | PROTEIN 204 (IFI-204). |
| Msa.3660.0 | W08473 | Homologous to sp P30306: M-PHASE INDUCER PHOSPHATASE 2 |
| | | (EC 3.1.3.48). |
| Msa.17097.0 | W98265 | Homologous to sp Q07120: ZINC FINGER PROTEIN GFI-1 |
| | | (GROWTH FACTOR INDEPENDENCE-1). |
| Msa.1615.0 | M36778 | Mouse GTP-binding protein alpha subunit (G0B-alpha) mRNA, |
| | | complete cds |
| Msa.1021.0 | M77678 | Mouse NKR-P1 (gene-40) mRNA, complete cds |
| Msa.28183.0 | AA068847 | Homologous to sp P30285: CELL DIVISION PROTEIN KINASE 4 |
| | | (EC 2.7.1.—) (PSK-J3). |
| Msa.23573.0 | AA050022 | Homologous to sp P10287: PLACENTAL-CADHERIN PRECURSOR |
| | | (P-CADHERIN). |
| Msa.803.0 | J00475 | Part of messenger RNA for mouse delta-immunoglobulin (codes for |
| | | part of exon 8 - one of two alternate C-termini) |
| Msa.2980.0 | M83219 | M. musculus intracellular calcium-binding protein (MRP14) mRNA, |
| | | complete cds |
| Msa.3605.0 | W67046 | Homologous to sp P14097: MACROPHAGE INFLAMMATORY |
| | | PROTEIN 1-BETA PREC |
| Msa.3234.0 | X97650 | M. musculus mRNA for myosin I |
| Msa.266.0 | M60493 | Mouse cystic fibrosis transmembrane conductance regulator (CFTR) |
| | | mRNA, complete cds |
| Msa.5481.0 | AA060106 | Homologous to sp P13928: ANNEXIN VIII (VASCULAR |
| | | ANTICOAGULANT-BETA) (VAC-BETA). |
| Msa.32014.0 | AA106256 | Homologous to sp P31945: NATURAL KILLER CELL ENHANCING |
| | | FACTOR B (NKEF-B). |
| Msa.3140.0 | U63841 | M. musculus neurogenic basic-helix-loop-helix protein (neuroD3) gene, |
| | | complete cds |
| Msa.35229.0 | AA119287 | Homologous to sp P04436: T-CELL RECEPTOR ALPHA CHAIN |
| | | PRECURSOR V REGION (HPB-MLT) (FRAGMENT). |
| Msa.2228.0_r_i | X60452 | M. musculus mRNA for cytochrome P-450IIIA |
| Msa.12766.0 | AA041634 | Homologous to sp P28659: BRAIN PROTEIN F41. |
| Msa.34650.0 | AA120463 | Homologous to sp P19971: THYMIDINE PHOSPHORYLASE (EC |
| | | 2.4.2.4) (PLATELET-DERIVED ENDOTHELIAL CELL GROWTH |
| | | FACTOR) (PD-ECGF) (GLIOSTATIN). |
| Msa.3019.0 | U58993 | M. musculus mSmad5 mRNA, complete cds |
| Msa.2541.0 | X72697 | M. musculus XMR mRNA |

TABLE 3
Altered gene expression in mouse ventral tegmental area due to cocaine
sensitization.

| Gene Name | Accession # | Gene ID |
|---|---|---|
| Msa.19779.0 | AA024297 | Homologous to sp Q01685: TRAM PROTEIN (TRANSLOCATING CHAIN- |
| | | ASSOCIATING MEMBRANE PROTEIN). 5′ similar to PIR: S30034 S30034 |
| | | translocating chain-associating membrane protein - human;, mRNA sequence |
| Msa.4753.0 | AA168362 | Homologous to sp P23458: TYROSINE-PROTEIN KINASE JAK1 (EC 2.7.1.112) |
| | | (JANUS KINASE 1). |
| Msa.3052.0 | U42384 | M. musculus fibroblast growth factor inducible gene 15 (FIN15) mRNA, complete cds |
| Msa.17539.0 | AA068302 | Homologous to sp P25388: GUANINE NUCLEOTIDE-BINDING PROTEIN BETA |
| | | SUBUNIT-LIKE PROTEIN 12.3 (P205) (RECEPTOR OF ACTIVATED PROTEIN |
| | | KINASE C 1) (RACK1). |
| Msa.25686.0 | AA060187 | Homologous to sp P26442: AUTOCRINE MOTILITY FACTOR RECEPTOR |
| | | PRECURSOR (AMP RECEPTOR) (GP78). |
| Msa.16618.0 | AA003990 | Homologous to sp P23152: PRE-MRNA SPLICING FACTOR SRP20 (X16 PROTEIN). |
| Msa.308.0_r | X74134 | Mus musculus ovalbumin upstream promoter transcription factor I COUP-TFI mRNA, |
| | | complete cds |
| Msa.11707.0 | AA145547 | Homologous to sp P48634: LARGE PROLINE-RICH PROTEIN BAT2 (HLA-B- |
| | | ASSOCIATED TRANSCRIPT 2). |
| Msa.16228.0 | W75523 | Homologous to sp P31007: LETHAL(1)DISCS LARGE-1 TUMOR SUPPRESSOR PRO |
| Msa.17332.0 | W89900 | Homologous to sp P36968: PHOSPHOLIPID HYDROPEROXIDE GLUTHATIONE |
| | | PEROXIDASE (EC 1.11.1.9) (PHGPX). |
| Msa.24485.0 | W89738 | Homologous to sp P20227: TRANSCRIPTION INITIATION FACTOR TFIID (TATA |
| Msa.308.0_i | X74134 | M. musculus ovalbumin upstream promoter transcription factor I COUP-TFI mRNA, |
| | | complete cds |
| Msa.6678.0 | W14673 | Homologous to sp P46379: LARGE PROLINE-RICH PROTEIN BAT3 (HLA-B- |
| | | ASSOCIATED TRANSCRIPT 3). |
| Msa.39525.0 | AA146375 | Homologous to sp P49186: STRESS-ACTIVATED PROTEIN KINASE JNK2 (EC 2.7.1.—) |
| | | (C-JUN N-TERMINAL KINASE 2) (SAPK-ALPHA) (P54-ALPHA). |
| Msa.11475.0 | W50352 | Homologous to sp P33124: LONG-CHAIN-FATTY-ACID-COA LIGASE, BRAIN |
| | | ISOZYME (EC 6.2.1.3) (LONG-CHAIN ACYL-COA SYNTHETASE) (LACS). |
| Msa.11623.0 | W50655 | Homologous to sp P28656: BRAIN PROTEIN DN38 (FRAGMENT). |
| Msa.1734.0 | W37000 | Mouse mRNA for monoclonal nonspecific suppressor factor beta, complete cds |
| Msa.927.0 | M21041 | Mouse microtubule-associated protein 2 (MAP2) mRNA, complete cds |
| Msa.5582.0 | W11746 | Homologous to sp P05215: TUBULIN ALPHA-4 CHAIN. |
| Msa.10274.0 | W46723 | Homologous to sp P07335: CREATINE KINASE, B CHAIN (EC 2.7.3.2). |
| Msa.1251.0 | M33385 | Mouse tyrosine protein kinase B (trkB) mRNA, complete cds |
| Msa.453.0 | M31690 | Mouse argininosuccinate synthetase (Ass) mRNA, complete cds |
| Msa.9135.0 | AA106492 | Homologous to sp P09456: CAMP-DEPENDENT PROTEIN KINASE TYPE I-ALPHA |
| | | REGULATORY CHAIN. |
| Msa.11817.0 | W50866 | Homologous to sp P06705: CALCINEURIN B SUBUNIT ISOFORM 1 (PROTEIN |
| | | PHOSPHATASE 2B REGULATORY SUBUNIT). |
| Msa.15338.0 | AA097366 | Homologous to sp Q00992: PUTATIVE REGULATORY PROTEIN TSC-22. |
| Msa.665.0 | M63659 | Mouse G-alpha-12 protein mRNA, complete cds |
| Msa.14942.0 | AA120109 | Homologous to sp P09912: INTERFERON-INDUCED PROTEIN 6-16 PRECURSOR |
| | | (IFI-6-16). |
| Msa.3062.0 | D87902 | Mouse mRNA for ARF5, complete cds |
| Msa.9761.0 | W41722 | Homologous to sp P11017: GUANINE NUCLEOTIDE-BINDING PROTEIN |
| | | G(I)/G(S)/G(T) BETA SUBUNIT 2 (TRANSDUCIN BETA CHAIN 2) (FRAGMENT). |
| Msa.10535.0 | AA162205 | Homologous to sp P27465: PHOSPHATIDYLSERINE DECARBOXYLASE |
| | | PROENZYME |
| Msa.7019.0 | AA163975 | Homologous to sp P10719: ATP SYNTHASE BETA CHAIN, MITOCHONDRIAL |
| | | PRECURSOR (EC 3.6.1.34). |
| Msa.10565.0 | AA020101 | Homologous to sp P28661: BRAIN PROTEIN H5. |

TABLE 4
Altered gene expression in mouse prefrontal cortex due to cocaine sensitization..

| Gene Name | Accession # | Gene ID |
|---|---|---|
| Msa.2192.0 | X52886 | Mouse mRNA for cathepsin D (EC 3.4.23.5) |
| Msa.2906.0_i | W13646 | Mouse mRNA for TI-225 |
| Msa.13479.0 | W57363 | Homologous to sp P32851: SYNTAXIN 1A (SYNAPTOTAGMIN |
| | | ASSOCIATED 35 KD PROTEIN) (P35A) (NEURON-SPECIFIC ANTIGEN |
| | | HPC-1). |
| Msa.29779.0 | AA087616 | Homologous to sp P25160: GTP-BINDING ADP-RIBOSYLATION FACTOR |
| | | HOMOLOG 1 PROTEIN. |
| Msa.29072.0 | AA073600 | Homologous to sp Q01485: ANKYRIN, BRAIN VARIANT 2 (ANKYRIN B) |
| | | (ANKYRIN, NONERYTHROID) (FRAGMENT). |
| Msa.2906.0_r_i | W13646 | Mouse mRNA for TI-225 |
| Msa.21996.0 | AA108956 | Homologous to sp Q04491: PROTEIN TRANSPORT PROTEIN SEC13. |
| Msa.2665.0 | X63039 | M. musculus RSP-1 mRNA for p33 protein |
| Msa.7151.0 | W17549 | Homologous to sp P18282: DESTRIN (ACTIN DEPOLYMERIZING FACTOR) |
| | | (ADF). |
| Msa.2582.0 | X60664 | Murine MPA gene for rod phosphodiesterase alpha-subunit |
| Msa.18213.0 | AA000227 | Homologous to sp Q09103: EYE-SPECIFIC DIACYLGLYCEROL KINASE |
| | | (EC 2.7.1.107) (RETINAL DEGENERATION A PROTEIN) (DIGLYCERIDE |
| | | KINASE) (DGK). |
| Msa.2254.0 | X77731 | M. musculus mRNA for Deoxycytidine kinase |
| Msa.3114.0 | Y08485 | M. musculus mRNA for synaptonemal complex protein |
| Msa.2005.0 | U51204 | M. musculus APC-binding protein EB2 mRNA, partial cds |
| Msa.2480.0 | X06305 | Mouse germ line TCR V-alpha F3.3 gene |

TABLE 5
Altered gene expression in mouse nucleus accumbens due to cocaine sensitization..

| Gene Name | Accession # | Gene ID |
|---|---|---|
| Msa.2447.0 | X00496 | Mouse Ia-associated invariant chain (Ii) mRNA fragment |
| Msa.2516.0 | X51683 | M. musculus T mRNA |
| Msa.1836.0 | X94353 | M. musculus cathelin related antimicrobial peptide, mRNA, complete |
| | | cds |
| Msa.1041.0 | M88355 | Mouse oxytocin-neurophysin I gene, complete cds |
| Msa.27917.0 | AA068062 | Homologous to sp P20111: ALPHA-ACTININ, SKELETAL MUSCLE |
| | | ISOFORM (F-ACTIN CROSS LINKING PROTEIN). |
| Msa.344.0 | U03723 | M. musculus AKR voltage-gated potassium-channel (KCNA4) gene, 5′ |
| | | region |
| Msa.10820.0 | W48968 | Homologous to sp P11980: PYRUVATE KINASE, M1 (MUSCLE) |
| | | ISOZYME (EC 2.7.1.40). |
| Msa.19580.0 | AA014024 | Homologous to sp P28023: DYNACTIN, 150 KD ISOFORM (150 KD |
| | | DYNEIN-ASSOCIATED POLYPEPTIDE) (DP-150) (DAP-150) (P150- |
| | | GLUED). |
| PyruCarbMur-MA | #N/A | PyruCarbMur-MA |

Table 6 identifies human genes in SHSY-5Y neuroblastoma cell cultures that have been shown to react by changes in mRNA expression levels in response to exposure to ethanol.

TABLE 6
Human genes or ESTs in SHSY-5Y neuroblastoma cell cultures that have been
shown to react by changes in mRNA expression levels in response to exposure to ethanol.

| Accession | Type | Name on chip | Description |
|---|---|---|---|
| D12620 | gene | 101D12620 | Human mRNA for cytochrome P-450LTBV. |
| D42041 | gene | 1573D42041 | Human mRNA (KIAA0088) for ORF (alpha- |
| | | | glucosidase-related), partial cds. |
| D90226 | gene | 44D90226 | Human mRNA for OSF-1. |
| H06695 | 3′ UTR | 7137H06695 | NEURONAL ACETYLCHOLINE RECEPTOR |
| | | | PROTEIN, ALPHA-2 CHAIN PRECURSOR (Rattus |
| | | | norvegicus) |
| H07142 | 3′ UTR | 1051H07142 | INTEGRIN ALPHA-6 PRECURSOR (Homo sapiens) |
| H11940 | 3′ UTR | 1043H11940 | X INACTIVE SPECIFIC TRANSCRIPT PROTEIN |
| | | | (Mus musculus) |
| H14506 | 3′ UTR | 1838H14506 | PRE-B CELL GROWTH STIMULATING FACTOR |
| | | | PRECURSOR (Mus musculus) |
| H15162 | 3′ UTR | 2425H15162 | MYOSIN HEAVY CHAIN 95F (Drosophila |
| | | | melanogaster) |
| H15417 | 3′ UTR | 2774H15417 | GLUTAMATE RECEPTOR 6 PRECURSOR (Rattus |
| | | | norvegicus) |
| H40677 | 3′ UTR | 8475H40677 | PROBABLE NUCLEAR ANTIGEN (Pseudorabies |
| | | | virus) |
| H56608 | 3′ UTR | 4152H56608 | SEX-DETERMINING TRANSFORMER PROTEIN 2 |
| | | | PRECURSOR (Caenorhabditis elegans) |
| H62556 | 3′ UTR | 4232H62556 | NUCLEOLIN (Mesocricetus auratus) |
| H64001 | 3′ UTR | 4252H64001 | CD9 ANTIGEN (Bos taurus) |
| H67849 | 3′ UTR | 4338H67849 | ALKALINE PHOSPHATASE, PLACENTAL TYPE 1 |
| | | | PRECURSOR (Homo sapiens) |
| H80543 | 3′ UTR | 2382H80543 | IG MU HEAVY CHAIN DISEASE PROTEIN |
| | | | (HUMAN);. |
| H82137 | 3′ UTR | 4473H82137 | PROTEIN PROSPERO (Drosophila melanogaster) |
| H84795 | 3′ UTR | 4515H84795 | 5-HYDROXYTRYPTAMINE 1B RECEPTOR (Homo |
| | | | sapiens) |
| H85111 | 3′ UTR | 4510H85111 | EBNA-2 NUCLEAR PROTEIN (Epstein-barr virus) |
| H87476 | 3′ UTR | 4551H87476 | ELONGATION FACTOR G, MITOCHONDRIAL |
| | | | PRECURSOR (Rattus norvegicus) |
| H88517 | 3′ UTR | 4562H88517 | ATP SYNTHASE A CHAIN (Trypanosoma brucei |
| | | | brucei) |
| H88787 | 3′ UTR | 2323H88787 | B-CELL LYMPHOMA 6 PROTEIN (Homo sapiens) |
| L21993 | gene | 2391L21993 | Human adenylyl cyclase mRNA, 3′ end of cds. |
| L28821 | gene | 7266L28821 | Homo sapiens alpha mannosidase II isozyme mRNA, |
| | | | complete cds. |
| L33881 | gene | 1935L33881 | Homo sapiens (EST02087-3) protein kinase C iota |
| | | | isoform, complete cds. |
| L41907 | gene | 4120L41907 | Homo sapiens retinoblastoma susceptibility protein |
| | | | (RB1) gene from tumor RBF29, exon 20, bases 156540-156889 |
| | | | in L11910. |
| M14083 | gene | 1881M14083 | Human beta-migrating plasminogen activator inhibitor I |
| | | | mRNA, 3′ end. |
| M15205 | gene | 2064M15205 | Human thymidine kinase gene, complete cds, with |
| | | | clustered Alu repeats in the introns. |
| M16938 | gene | 824M16938 | Human homeo box c8 protein, mRNA, complete cds. |
| M22995 | gene | 869M22995 | RAS-RELATED PROTEIN RAP-1A (HUMAN);. |
| M26683 | gene | 341M26683 | Human interferon gamma treatment inducible mRNA. |
| M27533 | gene | 842M27533 | Human Ig rearranged B7 protein mRNA VC1-region, |
| | | | complete cds. |
| M28622 | gene | 839M28622 | Human interferon beta-1 (IFN-beta-1) mRNA, complete |
| | | | cds. |
| M29065 | gene | 1043M29065 | Human hnRNP A2 protein mRNA. |
| M34057 | gene | 2055M34057 | TRANSFORMING GROWTH FACTOR BETA-1 |
| | | | BINDING PROTEIN (HUMAN); contains MER22 |
| | | | repetitive element;. |
| M38690 | gene | 1253M38690 | Human CD9 antigen mRNA, complete cds. |
| M58050 | gene | 2196M58050 | Human membrane cofactor protein (MCP) mRNA, |
| | | | complete cds. |
| M67466 | gene | 829M67466 | Human major 3-beta-hydroxysteroid |
| | | | dehydrogenase/delta-5-delta-4 isomerase mRNA, |
| | | | complete cds. |
| M77140 | gene | 1938M77140 | H. sapiens pro-galanin mRNA, 3′ end. |
| M81182 | gene | 1921M81182 | H. sapiens peroxisomal 70 kD membrane protein mRNA, |
| | | | complete cds. |
| M86699 | gene | 2083M86699 | Human kinase (TTK) mRNA, complete cds. |
| M94890 | gene | 1944M94890 | Human pregnancy-specific beta-1-glycoprotein 11 |
| | | | (PSG11) mRNA, complete cds. |
| M95787 | gene | 1626M95787 | SMOOTH MUSCLE PROTEIN 22-ALPHA |
| | | | (HUMAN); contains OFR repetitive element;. |
| M98331 | gene | 715M98331 | Homo sapiens defensin 6 mRNA, complete cds. |
| M99626 | gene | 91M99626 | Human Mid1 gene, partial cds. |
| M99701 | gene | 1931M99701 | Homo sapiens (pp21) mRNA, complete cds. |
| R08021 | 3′ UTR | 2156R08021 | INORGANIC PYROPHOSPHATASE (Bos taurus) |
| R15944 | 3′ UTR | 2338R15944 | PROTEIN TRANSLATION FACTOR SUI1 |
| | | | HOMOLOG (Arabidopsis thaliana) |
| R17909 | gene | 2307R17909 | 2-OXOISOVALERATE DEHYDROGENASE BETA |
| | | | SUBUNIT PRECURSOR (HUMAN);. |
| R26139 | 3′ UTR | 2050R26139 | TRANSCRIPTION INITIATION FACTOR IIB |
| | | | (HUMAN);. |
| R37964 | 3′ UTR | 1441R37964 | HEPARIN-BINDING EGF-LIKE GROWTH FACTOR |
| | | | PRECURSOR (Homo sapiens) |
| R38444 | 3′ UTR | 8178R38444 | TRANSCRIPTION FACTOR E2-ALPHA (Homo |
| | | | sapiens) |
| R43365 | 3′ UTR | 2394R43365 | 1-PHOSPHATIDYLINOSITOL-4,5-BISPHOSPHATE |
| | | | PHOSPHODIESTERASE GAMMA 1 (Homo sapiens) |
| R43532 | 3′ UTR | 2472R43532 | AGRIN PRECURSOR (Gallus gallus) |
| R45362 | 3′ UTR | 8743R45362 | ATP SYNTHASE A CHAIN (Trypanosoma brucei |
| | | | brucei) |
| R45687 | 3′ UTR | 2352R45687 | G2/MITOTIC-SPECIFIC CYCLIN G (Rattus |
| | | | norvegicus) |
| R48243 | gene | 2751R48243 | RAS-RELATED PROTEIN RHA1 (Arabidopsis |
| | | | thaliana) |
| R48492 | 3′ UTR | 277R48492 | H. sapiens NAP (nucleosome assembly protein) mRNA, |
| | | | complete cds. |
| R50499 | 3′ UTR | 9669R50499 | FIBRINOGEN BETA CHAIN PRECURSOR (Homo |
| | | | sapiens) |
| R52090 | 3′ UTR | 2674R52090 | GENERAL VESICULAR TRANSPORT FACTOR P115 |
| | | | (Bos taurus) |
| R54846 | 3′ UTR | 2822R54846 | BASIC FIBROBLAST GROWTH FACTOR |
| | | | RECEPTOR 1 PRECURSOR (Homo sapiens) |
| R54931 | 3′ UTR | 2825R54931 | DNA-DIRECTED RNA POLYMERASE II 13.6 KD |
| | | | POLYPEPTIDE (Saccharomyces cerevisiae) |
| R55687 | 3′ UTR | 1947R55687 | ASIALOGLYCOPROTEIN RECEPTOR 2 (Mus |
| | | | musculus) |
| R63621 | 3′ UTR | 2168R63621 | DEVELOPMENTAL PROTEIN SEVEN IN |
| | | | ABSENTIA (Drosophila melanogaster) |
| R71195 | 3′ UTR | 2973R71195 | RAS-RELATED PROTEIN RAB-2 (HUMAN);. |
| T49117 | 3′ UTR | 1231T49117 | ADRENAL SPECIFIC 30 KD PROTEIN (HUMAN). |
| T50769 | 3′ UTR | 9911T50769 | GOLIATH PROTEIN (Drosophila melanogaster) |
| T53412 | 3′ UTR | 1773T53412 | KERATIN, TYPE I CYTOSKELETAL 18 (HUMAN). |
| T54767 | 3′ UTR | 1052T54767 | SPARC PRECURSOR (Homo sapiens) |
| T55607 | 3′ UTR | 1066T55607 | NEUROVIRULENCE FACTOR (Herpes simplex virus) |
| T56807 | 3′ UTR | 1101T56807 | TAT-BINDING PROTEIN-1 (HUMAN). |
| T60155 | 3′ UTR | 1221T60155 | ACTIN, AORTIC SMOOTH MUSCLE (HUMAN);. |
| T61090 | 3′ UTR | 1167T61090 | ENDOGLIN PRECURSOR (Homo sapiens) |
| T70046 | 3′ UTR | 1006T70046 | ENDOTHELIAL ACTIN-BINDING PROTEIN (Homo |
| | | | sapiens) |
| T86928 | 3′ UTR | 2002T86928 | Homo sapiens ARL1 mRNA, complete cds. |
| T96325 | 3′ UTR | 1848T96325 | GOLIATH PROTEIN (Drosophila melanogaster) |
| U01828 | gene | 167U01828 | MICROTUBULE-ASSOCIATED PROTEIN 2 |
| | | | (HUMAN);. |
| U05237 | gene | 238U05237 | Human fetal Alz-50-reactive clone 1 (FAC1) mRNA, |
| | | | complete cds. |
| U11791 | gene | 516U11791 | Human cyclin H mRNA, complete cds. |
| U13044 | gene | 78U13044 | Human nuclear respiratory factor-2 subunit alpha |
| | | | mRNA, complete cds. |
| U14588 | gene | 2232U14588 | Human paxillin mRNA, complete cds. |
| U15655 | gene | 4128U15655 | Human ets domain protein ERF mRNA, complete cds. |
| U19178 | gene | 2257U19178 | Human (Hin-3)/HIV1 promoter region chimeric mRNA, |
| | | | complete cds. |
| U19523 | gene | 2326U19523 | Human GTP cyclohydrolase I mRNA, complete cds. |
| U19878 | gene | 2327U19878 | Human transmembrane protein mRNA, complete cds. |
| U20240 | gene | 2262U20240 | Human C/EBP gamma mRNA, complete cds. |
| U28368 | gene | 2113U28368 | Human Id-related helix-loop-helix protein Id4 mRNA, |
| | | | complete cds. |
| U29195 | gene | 3346U29195 | Human neuronal pentraxin II (NPTX2) gene, exon 5 and |
| | | | complete cds. |
| X02761 | gene | 2706X02761 | Human mRNA for fibronectin (FN precursor). |
| X05908 | gene | 2851X05908 | Human mRNA for lipocortin. |
| X12369 | gene | 3305X12369 | TROPOMYOSIN ALPHA CHAIN, SMOOTH |
| | | | MUSCLE (HUMAN);. |
| X13255 | gene | 2338X13255 | Human mRNA for dopamine beta-hydroxylase type a |
| | | | (EC 1.14.17.1). |
| X14787 | gene | 1117X14787 | Human mRNA for thrombospondin. |
| X16416 | gene | 1217X16416 | Human c-abl mRNA encoding p150 protein. |
| X51420 | gene | 2319X51420 | Human mRNA for tyrosinase-related protein. |
| X53586 | gene | 2821X53586 | Human mRNA for integrin alpha 6. |
| X55740 | gene | 1376X55740 | Human placental cDNA coding for 5′nucleotidase (EC |
| | | | 3.1.3.5). |
| X59798 | gene | 1366X59798 | Human PRAD1 mRNA for cyclin. |
| X60673 | gene | 2572X60673 | Human AK3 mRNA for adenylate kinase 3. |
| X62055 | gene | 1063X62055 | H. sapiens PTP1C mRNA for protein-tyrosine |
| | | | phosphatase 1C. |
| X70940 | gene | 2689X70940 | H. sapiens mRNA for elongation factor 1 alpha-2. |
| X74837 | gene | 2799X74837 | H. sapiens HUMM9 mRNA. |
| X78932 | gene | 2524X78932 | H. sapiens HZF9 mRNA for zinc finger protein. |
| X89066 | gene | 2405X89066 | H. sapiens mRNA for TRPC1 protein. |
| Y00067 | gene | 4123Y00067 | Human gene for neurofilament subunit M (NF-M). |
| Z19002 | gene | 4124Z19002 | H. sapiens of PLZF gene encoding kruppel-like zinc |
| | | | finger protein. |
| Z22936 | gene | 504Z22936 | H. sapiens TAP2E mRNA, complete CDS. |
| Z24727 | gene | 1130Z24727 | H. sapiens tropomyosin isoform mRNA, complete CDS. |
| Z38102 | gene | 2029Z38102 | H. sapiens mRNA for interleukin-11 receptor. |
| Z46629 | gene | 2355Z46629 | H. sapiens SOX9 mRNA. |

C) Identification of Homologous Genes and ESTs Whose Expression is Altered by Drugs of Abuse.

While in many instances the gene or EST identified in Tables 1-6 above is a mouse gene or EST, this invention also contemplates the use of homologous genes or ESTs from other species in the assays described herein. Thus, for example, where Tables 1-6 identify a mouse gene or EST, this invention contemplates the use of the human homologue as well as the homologues of other species, e.g. rabbit, horse, pig, goat, rat, etc.

Identification of suitable homologues is accomplished by routine search of the nucleic acid or protein databases. Thus, for example, one can enter the gene accession number in the by the National Center for Biotechnology Information (NCBI) Entrez browser (http://www.ncbi.nlm.nih.gov/Entrez/index.html) to perform a GenBank search for a given sequence. The database entry will identify known homologues. Alternatively, the sequence information can be entered and a BLAST search performed that will reveal other similar nucleic acid (or polypeptide) sequences. Preferred homologous sequences will share greater than 50%, preferably greater than 75%, more preferably greater than 80% and most preferably greater than 90% or 95% sequence identity with a gene or EST identified in Tables 1-6.

II. Assays of Expression Level(s) of the Genes and/or ESTs Identified Herein.

Assays of copy number or level of activity of one or more of the genes or ESTs identified herein provides a useful tool to screen for modulators of an organism's response to drugs of abuse, and/or to characterize an organism's response to such modulators or to particular drugs of abuse (e.g. opiates, cocaine, alcohol, etc.). Because the nucleic acid sequences of the various genes and ESTs identified herein are known, copy number and/or activity level can be directly measured according to a number of different methods as described below.

It will be recognized that expression levels of a gene can be altered by changes in the copy number of the gene, and/or by changes in the transcription of the gene product (i.e. transcription of mRNA), and/or by changes in translation of the gene product (i.e. translation of the protein), and/or by post-translational modification(s) (e.g. protein folding, glycosylation, etc.). Thus, it is possible to determine expression levels by a number of methods that involve assaying for copy number, level of transcribed mRNA, level of translated protein, activity of translated protein, etc. Examples of such approaches are, as described below.

A) Nucleic-Acid Based Assays.

1) Target Molecules.

As indicated above, gene expression can be varied by changes in copy number of the gene and/or changes in the regulation of gene expression. Changes in copy number are most easily detected by direct changes in genomic DNA, while changes in expression level can be detected by measuring changes in mRNA and/or a nucleic acid derived from the mRNA (e.g. reverse-transcribed cDNA, etc.).

In order to measure the nucleic acid concentration in a sample, it is desirable to provide a nucleic acid sample for such analysis. Where it is desired that the nucleic acid concentration, or differences in nucleic acid concentration between different samples, reflect transcription levels or differences in transcription levels of a gene or genes, it is desirable to provide a nucleic acid sample comprising mRNA transcript(s) of the gene or genes, or nucleic acids derived from the mRNA transcript(s). As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, suitable samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

In a particularly preferred embodiment, where it is desired to quantify the transcription level (and thereby expression) of a one or more genes in a sample, the nucleic acid sample is one in which the concentration of the mRNA transcript(s) of the gene or genes, or the concentration of the nucleic acids derived from the mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes. Where more precise quantification is required appropriate controls can be run to correct for variations introduced in sample preparation and hybridization as described herein. In addition, serial dilutions of “standard” target mRNAs can be used to prepare calibration curves according to methods well known to those of skill in the art. Of course, where simple detection of the presence or absence of a transcript or large differences of changes in nucleic acid concentration is desired, no elaborate control or calibration is required.

In the simplest embodiment, such a nucleic acid sample is the total mRNA or a total cDNA isolated and/or otherwise derived from a biological sample. The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

The nucleic acid (either genomic DNA or mRNA) may be isolated from the sample according to any of a number of methods well known to those of skill in the art. One of skill will appreciate that where alterations in the copy number of a gene are to be detected genomic DNA is preferably isolated. Conversely, where expression levels of a gene or genes are to be detected, preferably RNA (mRNA) is isolated.

Methods of isolating total mRNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993)).

In a preferred embodiment, the total nucleic acid is isolated from a given sample using, for example, an acid guanidinium-phenol-chloroform extraction method and polyA+ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified nucleic acid.

One preferred internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of radioactivity (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al., Academic Press, Inc. N.Y., (1990).

Other suitable amplification methods include, but are not limited to polymerase chain reaction (PCR) (Innis, et al., PCR Protocols. A guide to Methods and Application. Academic Press, Inc. San Diego, (1990)), ligase chain reaction (LCR) (see Wu and Wallace, Genomics, 4: 560 (1989), Landegren, et al., Science, 241: 1077 (1988) and Barringer, et al., Gene, 89: 117 (1990), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87: 1874 (1990)).

In a particularly preferred embodiment, the sample mRNA is reverse transcribed with a reverse transcriptase and a primer consisting of oligo dT and a sequence encoding the phage T7 promoter to provide single stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA. Methods of in vitro polymerization are well known to those of skill in the art (see, e.g., Sambrook, supra.) and this particular method is described in detail by Van Gelder, et al., Proc. Natl. Acad. Sci. USA, 87: 1663-1667 (1990) who demonstrate that in vitro amplification according to this method preserves the relative frequencies of the various RNA transcripts. Moreover, Eberwine et al. Proc. Natl. Acad. Sci. USA, 89: 3010-3014 provide a protocol that uses two rounds of amplification via in vitro transcription to achieve greater than 106 fold amplification of the original starting material thereby permitting expression monitoring even where biological samples are limited.

2) Hybridization-Based Assays.

i) Detection of Copy Number.

One method for evaluating the copy number of a genomic DNA or the encoding nucleic acid in a sample involves a Southern transfer. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid.

An alternative means for determining the copy number of a gene or EST of this invention is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

Preferred hybridization-based assays include, but are not limited to, traditional “direct probe” methods such as Southern blots or in situ hybridization (e.g., FISH), and “comparative probe” methods such as comparative genomic hybridization (CGH). The methods can be used in a wide variety of formats including, but not limited to substrate- (e.g. membrane or glass) bound methods or array-based approaches as described below.

In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained.

The probes are typically labeled, e.g., with radioisotopes or fluorescent reporters. Preferred probes are sufficiently long so as to specifically hybridize with the target nucleic acid(s) under stringent conditions. The preferred size range is from about 50 bp to about 1000 bases.

In some applications it is necessary to block the hybridization capacity of repetitive sequences. Thus, in some embodiments, tRNA, human genomic DNA, or Cot-1 DNA is used to block non-specific hybridization.

Another effective approach for the quantification of copy number og the gene(s) or EST(s) of this invention is comparative genomic hybridization. In this method, a first collection of (sample) nucleic acids (e.g. from a test sample derived from an organism, tissue, or cell exposed to one or more drugs of abuse) is labeled with a first label, while a second collection of (control) nucleic acids (e.g. from a normal “unexposed” organism, tissue, or cell) is labeled with a second label. The ratio of hybridization of the nucleic acids is determined by the ratio of the two (first and second) labels binding to each fiber in the array. Where there are chromosomal deletions or multiplications, differences in the ratio of the signals from the two labels will be detected and the ratio will provide a measure of the gene and/or EST copy number.

Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In Situ Hybridization Protocols, Choo, ed., Humana Press, Totowa, N.J. (1994), etc. In one particularly preferred embodiment, the hybridization protocol of Pinkel et al. (1998) Nature Genetics 20: 207-211, or of Kallioniemi (1992) Proc. Natl. Acad Sci USA 89:5321-5325 (1992) is used.

ii) Detection of Gene Transcript.

Methods of detecting and/or quantifying the transcript(s) of one or more gene(s) or EST(s) of this invention (e.g. mRNA or cDNA made therefrom) using nucleic acid hybridization techniques are known to those of skill in the art (see Sambrook et al. supra). For example, one method for evaluating the presence, absence, or quantity of gene or EST reverse-transcribed cDNA involves a Southern transfer as described above. Alternatively, in a Northern blot, mRNA is directly quantitated. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify and/or quantify the target mRNA.

The probes used herein for detection of the gene(s) and/or EST(s) of this invention can be full length or less than the full length of the gene or EST. Shorter probes are empirically tested for specificity. Preferably nucleic acid probes are 20 bases or longer in length. (see Sambrook et al. for methods of selecting nucleic acid probe sequences for use in nucleic acid hybridization.) Visualization of the hybridized portions allows the qualitative determination of the presence or absence of gene(s) and/or EST(s) of this invention.

3) Amplification-Based Assays.

In still another embodiment, amplification-based assays can be used to measure or level of gene (or EST) transcript. In such amplification-based assays, the target nucleic acid sequences act as template(s) in amplification reaction(s) (e.g. Polymerase Chain Reaction (PCR) or reverse-transcription PCR (RT-PCR)). In a quantitative amplification, the amount of amplification product will be proportional to the amount of template in the original sample. Comparison to appropriate (e.g. healthy tissue unexposed to drug(s) of abuse) controls provides a measure of the copy number or transcript level of the target gene or EST.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). The known nucleic acid sequence(s) for the genes and ESTs of this invention are available from GenBank using the information provided in Tables 1-6 is sufficient to enable one of skill to routinely select primers to amplify any portion of the gene.

Other suitable amplification methods include, but are not limited to ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

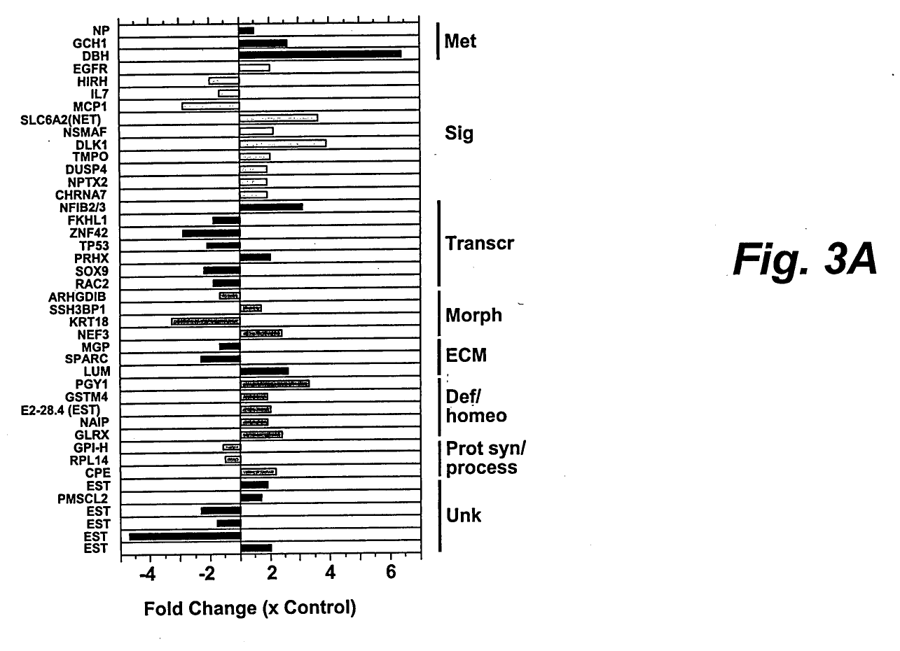

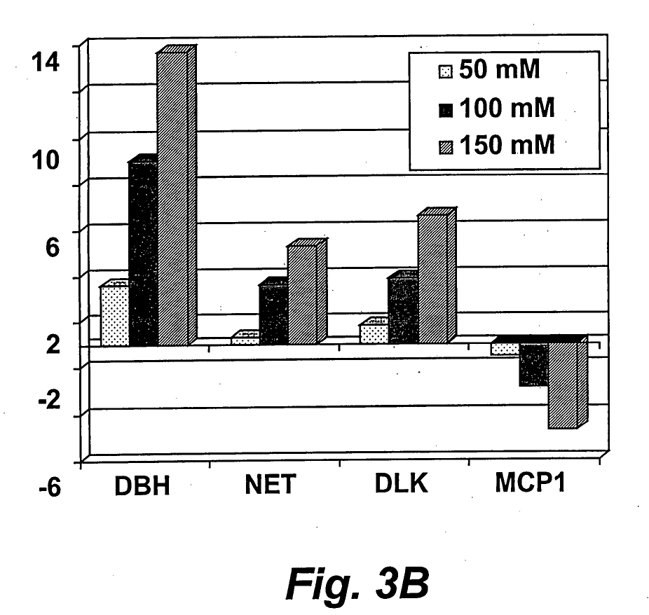

As indicated above, PCR assay methods are well known to those of skill in the art. Similarly, RT-PCR methods are also well known. Moreover, probes for such an RT-PCR assay are provided below in Table 1 and the assay is illustrated in Example 1 (see, e.g., FIG. 3).

4) Hybridization Formats and Optimization of Hybridization Conditions.

a) Array-Based Hybridization Formats.

The methods of this invention are particularly well suited to array-based hybridization formats. For a description of one preferred array-based hybridization system utilizing the Affymetrix GeneChip® system see Example 1.

Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

This simple spotting, approach has been automated to produce high density spotted arrays (see, e.g., U.S. Pat. No. 5,807,522). This patent describes the use of an automated system that taps a microcapillary against a surface to deposit a small volume of a biological sample. The process is repeated to generate high density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high density arrays is also described in U.S. Pat. Nos. 5,744,305, 5,800,992 and 5,445,934.

In brief, the light-directed combinatorial synthesis of oligonucleotide arrays on glass surfaces proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′-photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In a preferred embodiment, the arrays used in this invention can comprise either probe or target nucleic acids. These probes or target nucleic acids are then hybridized respectively with their “target” nucleic acids. Because the target gene and/or EST sequences listed in Tables 1-6 are known, oligonucleotide arrays can be synthesized containing one or multiple probes specific to any one or more of the genes and/or ESTs of this identified in invention.

In another embodiment the array, particularly a spotted array, can include genomic DNA, e.g. one or more clones that provide a high resolution scan of the genome containing the gene(s) and/or EST(s) of this invention. Such clones are available from commercial libraries. The nucleic acid clones can be obtained from, e.g., HACs, MACs, YACs, BACs, PACs, P1s, cosmids, plasmids, inter-Alu PCR products of genomic clones, restriction digests of genomic clones, cDNA clones, amplification (e.g., PCR) products, and the like.

In various embodiments, the array nucleic acids are derived from previously mapped libraries of clones spanning or including the sequences of the invention. The arrays can be hybridized with a single population of sample nucleic acid or can be used with two differentially labeled collections (as with a test sample and a reference sample).

Many methods for immobilizing nucleic acids on a variety of solid surfaces are known in the art. A wide variety of organic and inorganic polymers, as well as other materials, both natural and synthetic, can be employed as the material for the solid surface. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials which may be employed include paper, ceramics, metals, metalloids, semiconductive materials, cermets or the like. In addition, substances that form gels can be used. Such materials include, e.g., proteins (e.g., gelatins), lipopolysaccharides, silicates, agarose and polyacrylamides. Where the solid surface is porous, various pore sizes may be employed depending upon the nature of the system.

In preparing the surface, a plurality of different materials may be employed, particularly as laminates, to obtain various properties. For example, proteins (e.g., bovine serum albumin) or mixtures of macromolecules (e.g., Denhardt's solution) can be employed to avoid non-specific binding, simplify covalent conjugation, enhance signal detection or the like. If covalent bonding between a compound and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

For example, methods for immobilizing nucleic acids by introduction of various functional groups to the molecules is known (see, e.g., Bischoff (1987) Anal. Biochem., 164: 336-344; Kremsky (1987) Nucl. Acids Res. 15: 2891-2910). Modified nucleotides can be placed on the target using PCR primers containing the modified nucleotide, or by enzymatic end labeling with modified nucleotides. Use of glass or membrane supports (e.g., nitrocellulose, nylon, polypropylene) for the nucleic acid arrays of the invention is advantageous because of well developed technology employing manual and robotic methods of arraying targets at relatively high element densities. Such membranes are generally available and protocols and equipment for hybridization to membranes is well known.

Target elements of various sizes, ranging from 1 mm diameter down to 1 μm can be used. Relatively simple approaches capable of quantitative fluorescent imaging of 1 cm² areas have been described that permit acquisition of data from a large number of target elements in a single image (see, e.g., Wittrup (1994) Cytometry 16:206-213, Pinkel et al. (1998) Nature Genetics 20: 207-211).

Arrays on solid surface substrates with much lower fluorescence than membranes, such as glass, quartz, or small beads, can achieve much better sensitivity. Substrates such as glass or fused silica are advantageous in that they provide a very low fluorescence substrate, and a highly efficient hybridization environment. Covalent attachment of the target nucleic acids to glass or synthetic fused silica can be accomplished according to a number of known techniques (described above). Nucleic acids can be conveniently coupled to glass using commercially available reagents. For instance, materials for preparation of silanized glass with a number of functional groups are commercially available or can be prepared using standard techniques (see, e.g., Gait (1984) Oligonucleotide Synthesis: A Practical Approach, IRL Press, Wash., D.C.). Quartz cover slips, which have at least 10-fold lower autofluorescence than glass, can also be silanized.

Alternatively, probes can also be immobilized on commercially available coated beads or other surfaces. For instance, biotin end-labeled nucleic acids can be bound to commercially available avidin-coated beads. Streptavidin or anti-digoxigenin antibody can also be attached to silanized glass slides by protein-mediated coupling using e.g., protein A following standard protocols (see, e.g., Smith (1992) Science 258: 1122-1126). Biotin or digoxigenin end-labeled nucleic acids can be prepared according to standard techniques. Hybridization to nucleic acids attached to beads is accomplished by suspending them in the hybridization mix, and then depositing them on the glass substrate for analysis after washing. Alternatively, paramagnetic particles, such as ferric oxide particles, with or without avidin coating, can be used.

b) Other Hybridization Formats.

A variety of nucleic acid hybridization formats are known to those skilled in the art. For example, common formats include sandwich assays and competition or displacement assays. Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labelled probes or the like. Other labels include ligands that bind to labeled antibodies, fluorophores, chemi-luminescent agents, enzymes, and antibodies which can serve as specific binding pair members for a labeled ligand.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

c) Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

d) Labeling and Detection of Nucleic Acids.

In a preferred embodiment, the hybridized nucleic acids are detected by detecting one or more labels attached to the sample nucleic acids. The labels may be incorporated by any of a number of means well known to those of skill in the art. Means of attaching labels to nucleic acids include, for example nick translation, or end-labeling by kinasing of the nucleic acid and subsequent attachment (ligation) of a linker joining the sample nucleic acid to a label (e.g., a fluorophore). A wide variety of linkers for the attachment of labels to nucleic acids are also known. In addition, intercalating dyes and fluorescent nucleotides can also be used.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Suitable chromogens which can be employed include those molecules and compounds which absorb light in a distinctive range of wavelengths so that a color can be observed or, alternatively, which emit light when irradiated with radiation of a particular wave length or wave length range, e.g., fluorescers.

Desirably, fluorescers should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye can differ from the unbound dye. Therefore, when referring to the various wavelength ranges and characteristics of the dyes, it is intended to indicate the dyes as employed and not the dye which is unconjugated and characterized in an arbitrary solvent.

Fluorescers are generally preferred because by irradiating a fluorescer with light, one can obtain a plurality of emissions. Thus, a single label can provide for a plurality of measurable events.

Detectable signal can also be provided by chemiluminescent and bioluminescent sources. Chemiluminescent sources include a compound which becomes electronically excited by a chemical reaction and can then emit light which serves as the detectable signal or donates energy to a fluorescent acceptor. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label may be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe—CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

B) Polypeptide-Based Assays.

1) Assay Formats.

In addition to, or in alternative to, the detection of nucleic acid level(s), alterations in expression of the genes and/or EST(s) identified herein can be detected and/or quantified by detecting and/or quantifying the amount and/or activity of translated polypeptide.

Thus, for example, where function of an EST is unknown, the expressed sequence tag provides sufficient protein sequence that antibodies specific to that sequence can routinely be produced and utilized in immunoassays for quantification of the polypeptide product. Alternatively, the protein product itself can be directly detected, e.g. as described below.

Where the function/activity of the gene(s) or gene(s) labeled by particular EST(s) of this invention are known, one of ordinary skill in the art can detect and/or quantify changes in expression by detecting changes in the characteristic activity of the polypeptide encoded by that gene. Thus, for example, in a preferred embodiment, the respectively target gene(s) identified herein include DBH (dopamine β hydroxylase) an enzyme catalyzing the formation of NE, NET (sodium-dependent NE transporter), DLK (delta-like protein), and MCP-1 (monocyte chemoattractant peptide 1) and gene expression can be assayed by detecting and/or quantifying the characteristic activity of each protein, e.g. as described herein.

2) Detection of Expressed Protein

The polypeptide(s) encoded by the gene(s) and/or EST(s) of this invention can be detected and quantified by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one preferred embodiment, the polypeptide(s) are detected/quantified in an electrophoretic protein separation (e.g. a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of polypeptide(s) of this invention in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) and may be directly labeled or alternatively may be subsequently detected using labeled antibodies (e.g., labeled sheep anti-mouse antibodies) that specifically bind to the a domain of the antibody.

In a preferred embodiments, the polypeptide(s) encoded by gene(s) and/or EST(s) of this invention are detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7Edition.

Immunological binding assays (or immunoassays) typically utilize a “capture agent” to specifically bind to and often immobilize the analyte (in this case a polypeptide encoded by the gene(s) or EST(s) identified herein). In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

As indicated above, immunoassays for the detection and/or quantification of polypeptide(s) encoded by the gene(s) or EST(s) of this invention can take a wide variety of formats well known to those of skill in the art.

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one particularly preferred embodiment, the antibody is immobilized on a solid substrate. The amount of target polypeptide bound to the antibody may be determined either by measuring the amount of target polypeptide present in an polypeptide/antibody complex, or alternatively by measuring the amount of remaining uncomplexed polypeptide.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein, can be produced as described below.

3) Detection of Enzyme Activity.

In another embodiment, levels of gene expression/regulation are assayed by measuring the enzymatic activity of the polypeptide encoded by the respective gene(s). Thus, for example, the DBH, NET, DLK, and MCP-1 are identified herein as genes whose expression levels changed in a dose-dependent manner in response to ethanol and are therefore believe to represent important targets of ethanol. Expression of these genes can be assayed by detecting and/or quantifying the characteristic activity of each protein, e.g. as described below.

Expression levels (really activity levels in this case) can be evaluated by measuring the characteristic activities of these genes in a biological sample. Thus, for example, the DBH polypeptide activity can be assayed assayed using the artificial DBH substrate tyramine. Tyramine is converted by DBH to octopamine, which is the oxidized to parahydroxybenzaldehyde by sodium periodate. The oxidation is stopped by sodium metabisulfite. Parahydroxybenzaldehyde is then quantified by its absorbance at 330 nm in the UV.

DBH uses Cu as a cofactor. Hence, anything that chelates Cu (such as EDTA) kills the enzyme (unfortunately, irreversibly). So, for circulating DBH activity, the assay should be done on serum, or in plasma anticoagulated with heparin, though not EDTA.

The basic protocol for the assay is described by Nagatsu et al. (1972) Clinical Chem., 18(9): 980-983, and variants of the protocol are described in detail by O'Connor et al. (1979) Mol Pharmacol. 16: 529-538, Frigon et al. (1981) Molec Pharmacol. 19: 444-450, O'Connor et al. (1983) J Hypertension 1: 227-233; Sokoloff et al. (1985) J Neurochem 44: 441-450, Ziegler et al. 1990) Kidney International 37: 1357-1362, and references cited therein.

Similarly, the activity of NET, a sodium-dependent norephinephrine transporter can be assayed in a cell based system by measuring the uptake/release of labeled norepinephrine. Alternatively, the regulation of norepinephrine transporters (NETs) in vitro, can be assayed by measured the binding of the NET-selective ligand [³H]nisoxetine in cell homogenates (e.g., PC12 cells) after exposure of intact cells to drugs of abuse and/or potential modulators.

MCP-1, known as a chemokine produced during inflammatory responses by a wide variety of cells, is a chemoattractant for macrophages, and thus is readily assayed by its effect on target cells.

Assays for activity of the polypeptide products of other genes identified herein will be known to those of skill in the art.

4) Antibodies to Polypeptides Expressed by the Genes or ESTs Identified Herein.

Either polyclonal or monoclonal antibodies may be used in the immunoassays of the invention described herein. Polyclonal antibodies are preferably raised by multiple injections (e.g. subcutaneous or intramuscular injections) of substantially pure polypeptides or antigenic polypeptides into a suitable non-human mammal. The antigenicity of the target peptides can be determined by conventional techniques to determine the magnitude of the antibody response of an animal that has been immunized with the peptide. Generally, the peptides that are used to raise antibodies for use in the methods of this invention should generally be those which induce production of high titers of antibody with relatively high affinity for target polypeptides encoded by the genes or ESTs of this invention.

If desired, the immunizing peptide may be coupled to a carrier protein by conjugation using techniques that are well-known in the art. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g. a mouse or a rabbit).

The antibodies are then obtained from blood samples taken from the mammal. The techniques used to develop polyclonal antibodies are known in the art (see, e.g., Methods of Enzymology, “Production of Antisera With Small Doses of Immunogen: Multiple Intradermal Injections”, Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies see for example, Coligan, et al. (1991) Unit 9, Current Protocols in Immunology, Wiley Interscience).

Preferably, however, the antibodies produced will be monoclonal antibodies (“mAb's”). For preparation of monoclonal antibodies, immunization of a mouse or rat is preferred. The term “antibody” as used in this invention includes intact molecules as well as fragments thereof, such as, Fab and F(ab′)^{2′} which are capable of binding an epitopic determinant. Also, in this context, the term “mab's of the invention” refers to monoclonal antibodies with specificity for a polypeptide encoded by a gene or EST identified in Tables 1-5 herein.

The general method used for production of hybridomas secreting mAbs is well known (Kohler and Milstein (1975) Nature, 256:495). Briefly, as described by Kohler and Milstein the technique comprised isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody which bound to cancer cell lines.

Confirmation of specificity among mAb's can be accomplished using relatively routine screening techniques (such as the enzyme-linked immunosorbent assay, or “ELISA”) to determine the elementary reaction pattern of the mAb of interest.

It is also possible to evaluate an mAb to determine whether it has the same specificity as a mAb of the invention without undue experimentation by determining whether the mAb being tested prevents a mAb of the invention from binding to the target polypeptide isolated as described above. If the mAb being tested competes with the mAb of the invention, as shown by a decrease in binding by the mAb of the invention, then it is likely that the two monoclonal antibodies bind to the same or a closely related epitope. Still another way to determine whether a mAb has the specificity of a mAb of the invention is to preincubate the mAb of the invention with an antigen with which it is normally reactive, and determine if the mAb being tested is inhibited in its ability to bind the antigen. If the mAb being tested is inhibited then, in all likelihood, it has the same, or a closely related, epitopic specificity as the mAb of the invention.

Antibodies fragments, e.g. single chain antibodies (scFv or others), can also be produced/selected using phage display technology. The ability to express antibody fragments on the surface of viruses that infect bacteria (bacteriophage or phage) makes it possible to isolate a single binding antibody fragment from a library of greater than 10¹⁰ nonbinding clones. To express antibody fragments on the surface of phage (phage display), an antibody fragment gene is inserted into the gene encoding a phage surface protein (pIII) and the antibody fragment-pIII fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage bearing antigen binding antibody fragments can be separated from non-binding phage by antigen affinity chromatography (McCafferty et al. (1990) Nature, 348: 552-554). Depending on the affinity of the antibody fragment, enrichment factors of 20 fold-1,000,000 fold are obtained for a single round of affinity selection. By infecting bacteria with the eluted phage, however, more phage can be grown and subjected to another round of selection. In this way, an enrichment of 1000 fold in one round can become 1,000,000 fold in two rounds of selection (McCafferty et al. (1990) Nature, 348: 552-554). Thus even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment natural V_{H} and V_{L} repertoires present in human peripheral blood lymphocytes are were isolated from unimmunized donors by PCR. The V-gene repertoires were spliced together at random using PCR to create a scFv gene repertoire which is was cloned into a phage vector to create a library of 30 million phage antibodies (Id.). From this single “naive” phage antibody library, binding antibody fragments have been isolated against more than 17 different antigens, including haptens, polysaccharides and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628). Antibodies have been produced against self proteins, including human thyroglobulin, immunoglobulin, tumor necrosis factor and CEA (Griffiths et al. (1993) EMBO J. 12: 725-734). It is also possible to isolate antibodies against cell surface antigens by selecting directly on intact cells. The antibody fragments are highly specific for the antigen used for selection and have affinities in the 1 μM to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

It will also be recognized that antibodies can be prepared by any of a number of commercial services (e.g., Berkeley antibody laboratories, Bethyl Laboratories, Anawa, Eurogenetec, etc.).

III. Assay Optimization.

The assays of this invention have immediate utility in monitoring the response of a cell, tissue, or organism to exposure to drugs of abuse or for screening for agents that modulate the response of the cell, tissue or organism to such drugs of abuse. The assays of this invention can be optimized for use in particular contexts, depending, for example, on the source and/or nature of the biological sample and/or the particular drugs of abuse, and/or the analytic facilities available.

Thus, for example, while in one embodiment, all of the genes/ESTs identified in Tables 1-6 are screened, in other preferred embodiments, subsets of these genes or ESTS are screened. Thus, for example, Table 1 provides a particularly preferred set of genes/ESTs whose expression is altered by exposure to ethanol. Preferred subset of genes/ESTs for the assays of this invention exclude Chrna7, the α7 subunit of the neuronal acetylcholine receptor (nAChRα7).

Other preferred sets of genes/ESTs are represented by Tables 2-6. In various preferred embodiments, the screening will involve screening for expression of various combinations of these sets, subsets of these sets and subsets of these combinations of sets of the genes and/or ESTS. In preferred embodiments, assays will include at least one gene and/or EST, preferably at least 5 different genes and/or ESTs, more preferably at least 10 different genes and/or ESTs, most preferably at least 15 different genes and/or ESTs. Other preferred embodiments include at least 20, at least 30, at least 40, at least 50, at least 60, at least 100 or at least 200 genes and/or ESTs.

In one most preferred embodiment, the assays detect alterations in the expression utilize any one or more of the following: DBK, NET, MCP-1 and DLK.

In addition, assay formats can be selected and/or optimized according to the availability of equipment and/or reagents. Thus, for example, where commercial antibodies or ELISA kits are available it may be desired to assay protein concentration. Conversely, where it is desired to screen for modulators that alter transcription of one or more of the genes or ESTs identified herein, nucleic acid based assays are preferred.

Routine selection and optimization of assay formats is well known to those of ordinary skill in the art.

Assays of this invention are scored according to routine methods well known to those of skill in the art. In a preferred embodiment, quantitative assays of this invention level are deemed to show a positive result, e.g. elevated expression of one or more genes, when the measured protein or nucleic acid level is greater than the level measured or known for a control sample (e.g. either a level known or measured for a normal healthy cell, tissue or organism mammal of the same species not exposed to the drug of abuse and/or putative modulator (test agent), or a “baseline/reference” level determined at a different tissue and/or a different time for the same individual. In a particularly preferred embodiment, the assay is deemed to show a positive result when the difference between sample and “control” is statistically significant (e.g. at the 85% or greater, preferably at the 90% or greater, more preferably at the 95% or greater and most preferably at the 98% or greater confidence level).

IV. High Throughput Screening.

The assays of this invention are also amenable to “high-throughput” modalities. Conventionally, new chemical entities with useful properties (e.g., modulation of CNS plasticity in response to drugs of abuse) are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A) Combinatorial Chemical Libraries

Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

B) High Throughput Assays of Chemical Libraries.

Any of the assays for that modulate the response of the gene(s) or EST(s) identified herein are amenable to high throughput screening. As described above, having identified the nucleic acid whose expression is altered upon exposure to a drug of abuse, likely modulators either inhibit expression of the gene product, or inhibit the activity of the expressed protein. Preferred assays thus detect inhibition of transcription (i.e., inhibition of mRNA production) by the test compound(s), inhibition of protein expression by the test compound(s), or binding to the gene (e.g., gDNA, or cDNA) or gene product (e.g., mRNA or expressed protein) by the test compound(s). Alternatively, the assay can detect inhibition of the characteristic activity of the gene product or inhibition of or binding to a receptor or other transduction molecule that interacts with the gene product. High throughput assays for the presence, absence, or quantification of particular nucleic acids or protein products are well known to those of skill in the art. Similarly, binding assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configuarable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

V. Detection of Polymorphisms in One or More Genes and/or ESTs Whose Regulation is Altered in Cells Subject to a Drug of Abuse.

In another embodiment, having identified herein, genes and/or ESTs whose regulation is altered upon chronic exposure of an organism, tissue, or cell to one or more drugs of abuse, it is desirable to evaluate how these genes or ESTs vary in natural populations. In particular, it is believed that various polymorphisms of these genes or ESTs could predispose an individual to tolerance of and/or addiction to one or more drugs of abuse, or conversely, other polymorphisms can reduce the development of tolerance and/or addiction to one or more drugs of abuse. Identification of such polymorphisms provides valuable markers that can be used in evaluating various treatment modalities and risk factors for epidemiological and other evaluations.

A wide variety of methods can be used to identify specific polymorphisms. For example, repeated sequencing of genomic material from large numbers of individuals, although extremely time consuming, can be used to identify such polymorphisms. Alternatively, ligation methods may be used, where a probe having an overhang of defined sequence is ligated to a target nucleotide sequence derived from a number of individuals. Differences in the ability of the probe to ligate to the target can reflect polymorphisms within the sequence. Similarly, restriction patterns generated from treating a target nucleic acid with a prescribed restriction enzyme or set of restriction enzymes can be used to identify polymorphisms. Specifically, a polymorphism may result in the presence of a restriction site in one variant but not in another. This yields a difference in restriction patterns for the two variants, and thereby identifies a polymorphism.

In a related method, polymorphisms can be identified using type-IIs endonucleases to capture ambiguous base sequences adjacent the restriction sites, and characterizing the captured sequences on oligonucleotide arrays. The patterns of these captured sequences are compared from various individuals, the differences being indicative of potential polymorphisms.

In one preferred embodiment, polymorphisms are screened using nucleic acid array-based methodologies, e.g., as described in U.S. Pat. No. 5,858,659 and in PCT publications WO 09909218 A1, WO 09905324 A1, WO 09856954 A1, and WO 09830883 A2.

In one embodiment, this is accomplished using arrays of oligonucleotide probes. These arrays may generally be “tiled” for a large number of specific polymorphisms. By “tiling” is generally meant the synthesis of a defined set of probes which is made up of a sequence complementary to the target sequence of interest, as well as preselected variations of that sequence, e.g., substitution of one or more given positions with one or more members of the basis set of monomers, i.e. nucleotides. Tiling strategies are discussed in detail in Published PCT Application No. WO 95/11995.

In a particular aspect, arrays are tiled for a number of specific, identified polymorphic marker sequences. In particular, the array is tiled to include a number of detection blocks, each detection block being specific for a specific polymorphic marker or set of polymorphic markers. For example, a detection block may be tiled to include a number of probes which span the sequence segment that includes a specific polymorphism. To ensure probes that are complementary to each variant, the probes are synthesized in pairs differing at the biallelic base.

In addition to the probes differing at the biallelic bases, monosubstituted probes are also generally tiled within the detection block. These monosubstituted probes have bases at and up to a certain number of bases in either direction from the polymorphism, substituted with the remaining nucleotides (selected from A, T, G, C or U). Typically, the probes in a tiled detection block will include substitutions of the sequence positions up to and including those that are 5 bases away from the base that corresponds to the polymorphism. Preferably, bases up to and including those in positions 2 bases from the polymorphism will be substituted. The monosubstituted probes provide internal controls for the tiled array, to distinguish actual hybridization from artifactual cross-hybridization.

A variety of tiling configurations may also be employed to ensure optimal discrimination of perfectly hybridizing probes. For example, a detection block may be tiled to provide probes having optimal hybridization intensities with minimal cross-hybridization. For example, where a sequence downstream from a polymorphic base is G-C rich, it could potentially give rise to a higher level of cross-hybridization or “noise,” when analyzed. Accordingly, one can tile the detection block to take advantage of more of the upstream sequence. Optimal tiling configurations may be determined for any particular polymorphism by comparative analysis

Once an array is appropriately tiled for a given polymorphism or set of polymorphisms, the target nucleic acid is hybridized with the array and scanned. Hybridization and scanning are generally carried out by methods described in, e.g., Published PCT Application Nos. WO 92/10092 and WO 95/11995, and U.S. Pat. No. 5,424,186. In brief, a target nucleic: acid sequence which includes one or more previously identified polymorphic markers is amplified by well known amplification techniques, e.g., PCR. Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

VI. Arrays for Monitoring or Detecting Alterations of Gene Expression in Response to One or More Drugs of Abuse.

In another embodiment, this invention provides nucleic acid arrays for monitoring or detecting alterations gene expression in response to one or more drugs of abuse or for screening test agents for modulators of a cells, tissue's or organism's response to one or more drugs of abuse. In preferred embodiments, the arrays comprise one or more nucleic acid probes that hybridize specifically to nucleic acids comprising the ESTs or genes identified in Tables 1-6 or to human homologues of those genes or ESTs.

Preferred arrays predominantly comprise probes that are specific to the genes or ESTs identified in Tables 1-6 or to human homologues of the genes or ESTs listed in Tables 1-6. When referring to arrays that predominantly comprise probes to particular targets, it is intended to mean that of the target specific probes in an array (i.e., the probes in an array other than control probes (e.g. mismatch controls) and probes to housekeeping genes) more than 50%, preferably 60% or more, more preferably 80% or more, and most preferably 90%, or 95% or more are specific to the particular targets.

Thus, for example, if an array consisted of 100 probes specific to genes of Table 1, 100 mismatch control probes (i.e. one mismatch for each target specific probe) 100 control probes specific to housekeeping genes and 100 mismatch control probes for each control probe, for a total of 400 probes, the array would be said to predominantly comprise probes specific to genes of Table 1 if 51 or more (i.e., greater than 50% of the target-specific probes) probes of the array were specific to genes of Table 1 even though 51 probes only amount to about 25% of the total number of probes on the array.

The arrays can be high density arrays (e.g. having a probe density greater than 1000 probes/cm²) or relatively low-density (e.g. conventional dot blots). Also, as described above, the arrays can be arrays of synthetic oligonucleotides, synthesized in place, or can be spotted arrays of oligonucleotides, cDNAs, genomic DNAs, RNAs and the like.

Preferred arrays will include probes specific to at least one gene and/or EST, preferably at least 5 different genes and/or ESTs, more preferably at least 10 different genes and/or ESTs, most preferably at least 15 different genes and/or ESTs in Tables 1-6 (optionally excluding the α7 subunit of the neuronal acetylcholine receptor (nAChRα7)). Other preferred embodiments include probes specific to at least 20, at least 30, at least 40, at least 50, at least 60, at least 100 or at least 200 genes and/or ESTs of Tables 1-6 (optionally excluding the α7 subunit of the neuronal acetylcholine receptor (nAChRα7)).

Particularly preferred arrays comprise at least 1,000, preferably at least 2,000, more preferably at least 5,000, and most preferably at least 10,000, at least about 20,0000, at least about 30,000, or even at least about 50,000 or 100,000 probes to different genes. The arrays can have probe densities greater than 500 probes/cm², preferably greater than about 1,000 different probes/cm², more preferably greater than about 2,000 different probes/cm², and most preferably greater than about 5,000 different probes/cm², or greater than about 10,000 different probes/cm², or even greater than about 20,000, greater than about 30,000, greater than about 50,000 or greater than about 100,000 different probes/cm². Preferred probe lengths are greater than about 10 nucleotides, preferably greater than about 20 nucleotides, more preferably greater than about 30 nucleotides, and most preferably greater than about 50, 100, 250 or even 500 nucleotides. In certain embodiments probe length is essentially unlimited (e.g. limited only to the length of the available nucleic acid(s), clones, etc.). In some embodiments, the probe(s) have a maximum length less than about 100,000 nucleotides, preferably less than about 50,000 nucleotides, more preferably less than about 10,000 nucleotides, and most preferably less than about 5, 000 or less than about 1,000, less than about 500, less than about 100, or less than about 50 nucleotides.

VII. Kits for Monitoring or Detecting Alterations of Gene Expression in Response to One or More Drugs of Abuse.

In another embodiment, this invention provides kits for monitoring or detecting alterations gene expression in response to one or more drugs of abuse or for screening test agents for modulators of a cells, tissue's or organism's response to one or more drugs of abuse. The kits comprise one or more of the nucleic acid arrays described herein and/or individual probes (labeled or unlabeled) specific for the gene(s) and/or ESTs identified in Tables 1-6, and/or one or more antibodies specific for polypeptides encoded by the genes and/or ESTs of Tables 1-6. Kits may optionally include any reagents and/or apparatus to facilitate practice of the assays described herein. Such reagents include, but are not limited to buffers, labels, labeled antibodies, labeled nucleic acids, filter sets for visualization of fluorescent labels, blotting membranes, and the like.

In addition, the kits may include instructional materials containing directions (i.e., protocols) for the practice of the assay methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

VIII. Design of Antagonists of Expression for Screening as Test Agents in the Assays Described Herein.

This invention provides methods of screening test agents for the ability to modulate (e.g. up-regulate or down-regulate) the expression of one or more of the genes and/or ESTs of Tables 1-6. While there is essentially no limit on the agents that may be tested according to the methods of this invention, in some embodiments, “rational” drug design principles can be utilized to enhance the likelihood of identifying effective test agents. Thus, for example, knowing the identity of the gene(s) or ESTs whose activity is to be altered/modulated, one can design classes of molecules that specifically interact with these genes and/or their promoters or other regulatory elements in the pathways associated with these genes.

Thus for example, potential antagonists of these genes or gene products include antibodies or, in some cases, oligonucleotides that bind to either the nucleic acid or the protein product of the gene or EST. Other potential antagonists also include proteins which are closely related to the protein products of the genes or ESTs identified herein, i.e. a fragment of the protein (e.g. a fragment of DBH), which has lost biological function and, when binding to its cognate target, elicits no response.

Other potential antagonists include an antisense constructs prepared through the use of antisense technology. Antisense technology can be used to control gene expression through triple-helix formation or antisense DNA or RNA, both methods of which are based on binding of a polynucleotide to DNA or RNA. For example, the 5′ coding portion of the polynucleotide sequence, which encodes for the mature polypeptides of the present invention, is used to design an antisense RNA oligonucleotide of from about 10 to 40 base pairs in length. A DNA oligonucleotide is designed to be complementary to a region of the gene involved in transcription (triple helix—see Lee et al., (1979) Nucl Acids Res. 6: 3073; Cooney et al., (1988) Science 241: 456; and Dervan et al., (1991) Science, 251: 1360), thereby preventing transcription and production of the polypeptide. The antisense RNA oligonucleotide hybridizes to the mRNA in vivo and blocks translation of the mRNA molecule into the polypeptide (antisense—see Okano (1991) J Neurochem., 56: 560; Oligodeoxynucleotides as Antisense Inhibitors of Gene Expression, CRC Press, Boca Raton, Fla. (1988)). The oligonucleotides described above can also be delivered to cells such that the antisense RNA or DNA is expressed in vivo to inhibit production of the target polypeptide(s).

Another potential antagonist is a small molecule which binds to the target polypeptide, making it inaccessible to ligands such that normal biological activity is prevented. Examples of small molecules include, but are not limited to, small peptides or peptide-like molecules.

Other potential antagonists include ribozymes that specifically target and cleave the mRNA(s) transcribed from the gene(s) or EST(s) identified herein. Ribozymes are RNA molecules having an enzymatic activity which is able to cleave and splice other separate RNA molecules in a nucleotide base sequence specific manner. Such enzymatic RNA molecules can be targeted to virtually any RNA transcript, and efficient cleavage and splicing achieved in vitro (Kim et al., (1987) Proc. Natl. Acad. Sci. USA, 84: 8788, Hazeloff et al. (1988) Nature, 234: 585, Cech (1988) JAMA, 260: 3030, and Jefferies et al. (1989) Nucleic Acid Res. 17: 1371).

IX. Expression of Genes and Polypeptides.

In some instances it is desired to express the protein products of the genes or ESTs identified herein either for use in generating antibodies or mimetics or in a therapeutic context where the organism is deficient in one or more of these proteins. Thus, in one embodiment, the present invention relates to vectors which contain polynucleotides of the present invention, host cells which are genetically engineered with vectors of the invention and the production of polypeptides of the invention by recombinant techniques.

In a preferred embodiment, the protein(s) of this invention or subsequences, are synthesized using recombinant DNA methodology. Generally this involves creating a DNA sequence that encodes the protein, placing the DNA in an expression cassette under the control of a particular promoter, expressing the protein in a host, isolating the expressed protein and, if required, renaturing the protein.

DNA encoding the proteins, protein subunits, or subsequences of this invention can be prepared by any suitable method as described above, including, for example, cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066.

Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences may be obtained by the ligation of shorter sequences.

Alternatively, subsequences may be cloned and the appropriate subsequences cleaved using appropriate restriction enzymes. The fragments may then be ligated to produce the desired DNA sequence.

In one embodiment, the proteins of this invention can be cloned using DNA amplification methods such as polymerase chain reaction (PCR). Thus, for example, the nucleic acid sequence or subsequence is PCR amplified, using a sense primer containing one restriction site (e.g., NdeI) and an antisense primer containing another restriction site (e.g., HindIII). This will produce a nucleic acid encoding the desired protein(s) having terminal restriction sites. This nucleic acid can then be easily ligated into a vector containing a nucleic acid encoding the second molecule and having the appropriate corresponding restriction sites.

Suitable PCR primers can be determined by one of skill in the art using the sequence information. Appropriate restriction sites can also be added to the nucleic acid encoding proteins by site-directed mutagenesis. The plasmid containing the protein-encoding nucleic acid is cleaved with the appropriate restriction endonuclease and then ligated into the vector encoding the second molecule according to standard methods.

The nucleic acid sequences encoding the desired protein(s) may be expressed in a variety of host cells, including E. coli, other bacterial hosts, yeast, and various higher eukaryotic cells such as the COS, CHO and HeLa cells lines and myeloma cell lines. As the protein(s) identified herein are typically found in eukaryotes, a eukaryote host is preferred. The recombinant protein gene will be operably linked to appropriate expression control sequences for each host. For E. coli this includes a promoter such as the T7, trp, or lambda promoters, a ribosome binding site and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences.

The plasmids of the invention can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant the proteins can be purified according to standard procedures of the art, including ammonium sulfate precipitation, affinity columns, column chromatography, gel electrophoresis and the like (see, generally, R. Scopes, (1982) Protein Purification, Springer-Verlag, N.Y.; Deutscher (1990) Methods in Enzymology Vol. 182: Guide to Protein Purification., Academic Press, Inc. N.Y.). Substantially pure compositions of at least about 90 to 95% homogeneity are preferred, and 98 to 99% or more homogeneity are most preferred. Once purified, partially or to homogeneity as desired, the polypeptides may then be used (e.g., as immunogens for antibody production).

One of skill in the art would recognize that after chemical synthesis, biological expression, or purification, the protein (s) may possess a conformation substantially different than the native conformations of the constituent polypeptides. In this case, it may be necessary to denature and reduce the polypeptide and then to cause the polypeptide to re-fold into the preferred conformation. Methods of reducing and denaturing proteins and inducing re-folding are well known to those of skill in the art (see, Debinski et al. (1993) J. Biol. Chem., 268: 14065-14070; Kreitman and Pastan (1993) Bioconjug. Chem., 4: 581-585; and Buchner, et al., (1992) Anal. Biochem., 205: 263-270). Debinski et al., for example, describes the denaturation and reduction of inclusion body proteins in guanidine-DTE. The protein is then refolded in a redox buffer containing oxidized glutathione and L-arginine.

One of skill would recognize that modifications can be made to the proteins without diminishing their biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

X. Administration of Modulators.

The compounds that supplement and/or modulate (e.g. downregulate) activity of the genes or ESTs identified herein can be administered by a variety of methods including, but not limited to parenteral, topical, oral, or local administration, such as by aerosol or transdermally, for prophylactic and/or therapeutic treatment. The pharmaceutical compositions can be administered in a variety of unit dosage forms depending upon the method of administration. For example, unit dosage forms suitable for oral administration include powder, tablets, pills, capsules and lozenges. It is recognized that the modulators (e.g. antibodies, antisense constructs, ribozymes, small organic molecules, etc.) when administered orally, must be protected from digestion. This is typically accomplished either by complexing the molecule(s) with a composition to render it resistant to acidic and enzymatic hydrolysis or by packaging the molecule(s) in an appropriately resistant carrier such as a liposome. Means of protecting agents from digestion are well known in the art.

The compositions for administration will commonly comprise a modulator dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs.

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art and are described in more detail in such publications as Remington's Pharmaceutical Science, 15th ed., Mack Publishing Company, Easton, Pa. (1980).

The compositions containing modulators of CYP24 can be administered for therapeutic or prophylactic treatments. In therapeutic applications, compositions are administered to a patient suffering from a disease (e.g., an epithelial cancer) in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the agents of this invention to effectively treat the patient.

XI. Gene Therapy.

In some instances it is expected that the pathological response of an organism to one or more drugs of abuse will reflect an imbalance or inadequacy in the response of one or more of the genes and/or ESTs identified herein. Such a response may be mitigated by compensatign for inadeqate regulation of the target gene. The genes and proteins associated with CNS response to drugs of abuse may be employed in accordance with the present invention by expression of such polypeptides in treatment modalities often referred to as “gene therapy.”

Thus, for example, cells from a patient may be engineered with a polynucleotide (e.g. a polynucleotide of Tables 1-6 and/or human homologues thereof), such as a DNA or RNA, to encode a polypeptide ex vivo. The engineered cells can then be provided to a patient to be treated with the polypeptide. In this embodiment, cells may be engineered ex vivo, for example, by the use of a retroviral plasmid vector containing RNA encoding a polypeptide of the present invention. Such methods are well-known in the art and their use in the present invention will be apparent from the teachings herein.

Similarly, cells may be engineered in vivo for expression of a polypeptide in vivo by procedures known in the art. For example, a polynucleotide of the invention may be engineered for expression in a replication defective retroviral vector. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a patient for engineering cells in vivo and expression of the polypeptide in vivo. These and other methods for administering a polypeptide of the present invention should be apparent to those skilled in the art from the teachings of the present invention.

Retroviruses from which the retroviral plasmid vectors herein above mentioned may be derived include, but are not limited to, Moloney Murine Leukemia Virus, Spleen Necrosis Virus, Rous Sarcoma Virus, Harvey Sarcoma Virus, Avian Leukosis Virus, Gibbon Ape Leukemia Virus, Human Immunodeficiency Virus, Adenovirus, Myeloproliferative Sarcoma Virus, and Mammary Tumor Virus. In a preferred embodiment, the retroviral plasmid vector is derived from Moloney Murine Leukemia Virus.

Such vectors will include one or more promoters for expressing the polypeptide. Suitable promoters which may be employed include, but are not limited to, the retroviral LTR; the SV40 promoter; and the human cytomegalovirus (CMV) promoter described in Miller et al. (1989) Biotechniques, 7: 980-990. Cellular promoters such as eukaryotic cellular promoters including, but not limited to, the histone, RNA polymerase III, and .beta.-actin promoters can also be used. Additional viral promoters which may be employed include, but are not limited to, adenovirus promoters, thymidine kinase (TK) promoters, and B19 parvovirus promoters. The selection of a suitable promoter will be apparent to those skilled in the art from the teachings contained herein.

The nucleic acid sequence encoding the polypeptide of the present invention will be placed under the control of a suitable promoter. Suitable promoters which may be employed include, but are not limited to, adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs (including the modified retroviral LTRs herein above described); the .beta.-actin promoter; and human growth hormone promoters. The promoter may also be the native promoter which controls the gene encoding the polypeptide.

The retroviral plasmid vector is employed to transduce packaging cell lines to form producer cell lines. Examples of packaging cells which may be transfected include, but are not limited to, the PE501, PA317, Y-2, Y-AM, PA12, T19-14×, VT19-17-1H2, YCRE, YCRIP, GP+E-86, GP+envAm12, and DAN cell lines as described in Miller, A., Human Gene Therapy, 1990, 1: 5-14. The vector may be transduced into the packaging cells through any means known in the art. Such means include, but are not limited to, electroporation, the use of liposomes, and CaPO.sub.4 precipitation. In one alternative, the retroviral plasmid vector may be encapsulated into a liposome, or coupled to a lipid, and then administered to a host.

The producer cell line will generate infectious retroviral vector particles, which include the nucleic acid sequence(s) encoding the polypeptides. Such retroviral vector particles may then be employed to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

### EXAMPLES

The following examples are offered to illustrate, but not to limit the claimed invention.

### Example 1

### Effects of Cocaine Sensitization on Mice

Mice were treated with intra peritoneal injection of cocaine (10 mg.kg) or saline every other day for up to 12 days such that they developed cocaine sensitization. Sensitization refers to an increase in locomotor activity that occurs following repeated exposure to drugs of abuse. Sensitization is stable for long periods of drug abstinence and thus clearly represents a plasticity that generates an increased CNS response to abused drugs as seen with addiction.

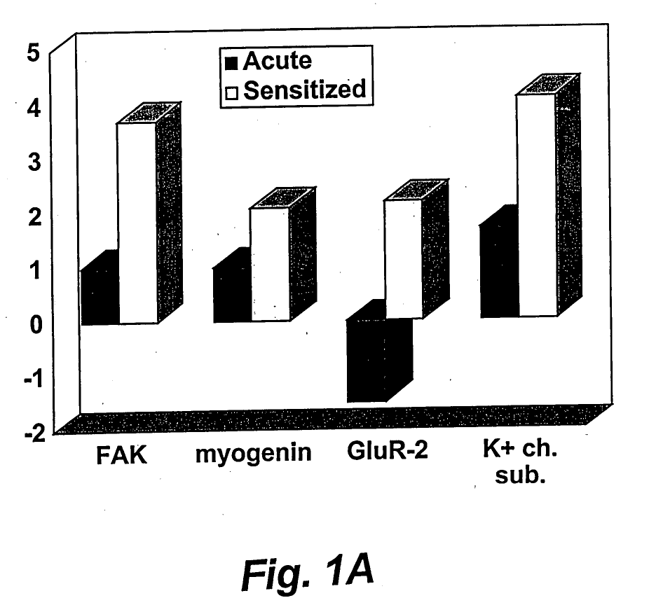

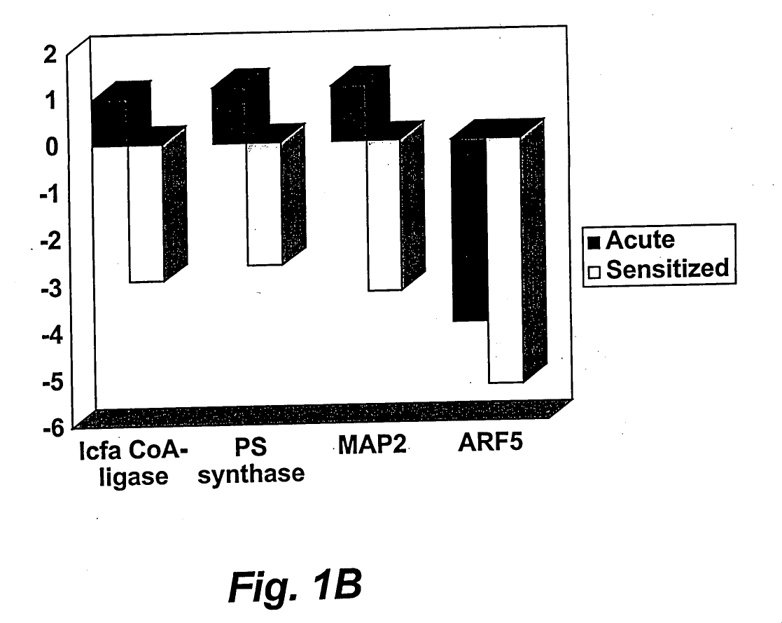

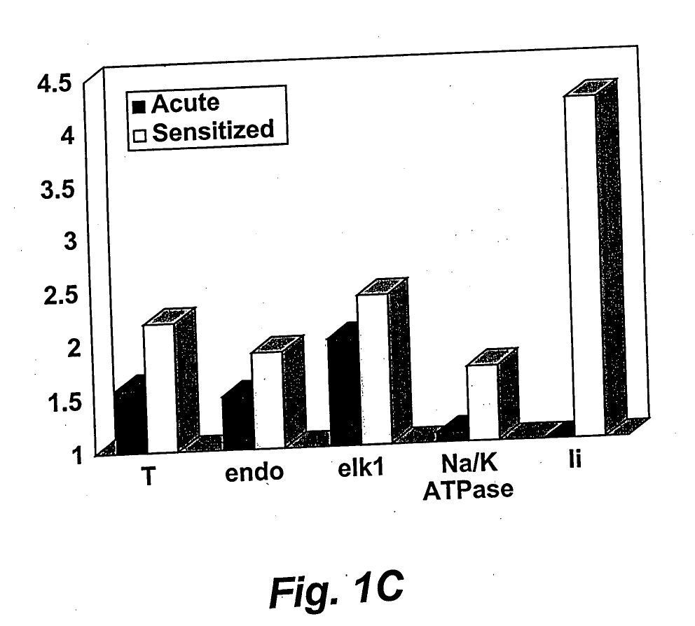

Behavioral testing for locomoter activity was done on each injection day. The results are illustrated in Tables 2-5 and in FIGS. 1A (VTA inductions), 1B (VTA inductions) and 1C (Nucleus Accumbens). Acute treatment was a single dose of cocaine.

As shown in FIG. 1A, FAK, myogenin, and K+ch. sub. all result in increased VTA inductions both by acute and sensitized exposure to cocaine, while GluR-2 demonstrates an increase in VTA induction after sensitized exposure but a decreased induction after acute exposure.

FIG. 1B depicts VTA inductions of cocaine sensitization genes. Icfa CoA-ligase, PS synthase and MAP2 all show increased induction after acute exposure and decreased induction after sensitized exposure. ARF 5 shows decreased induction during both acute and sensitized exposure.

FIG. 1C gene expression of cocaine sensitization genes in the nucleus accumbens region of the brain. For T, endo, elk1, Na/K ATPase and Li, sensitized exposure always resulted in much higher expression than acute exposure.

Table 7 depicts the results of DNA array analysis of gene expression in cocaine sensitization. Columns 3-6 show the number of genes with a greater than 2-fold change in response to acute (columns 3 and 4) or sensitized (columns 5 and 6) exposure. Columns 3 and 5 represent increases in gene expression, and columns 4 and 6 represent decreases in gene expression.

TABLE 7
DNA array analysis of gene expression in cocaine sensitization.

| | # Genes | Acute | | Sensitized | |
|---|---|---|---|---|---|
| Region | Detected | Increase | Decrease | Increase | Decrease |
| VTA | 3451 | 7 | 28 | 14 | 30 |
| NA | 3778 | 2 | 2 | 11 | 12 |
| PFC | 3703 | 1 | 24 | 25 | 7 |

### Example 2

### Effect(s) of Ethanol on SHSY-5Y Cells in Culture—Study 1

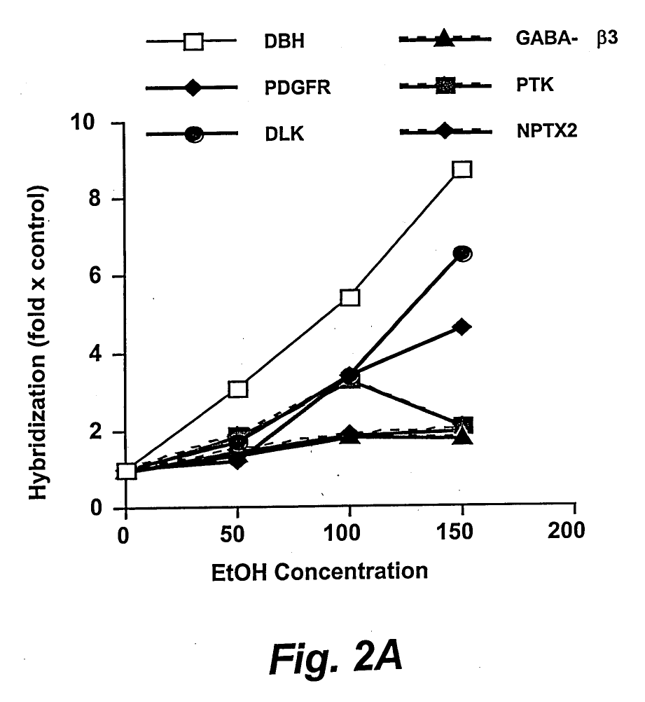

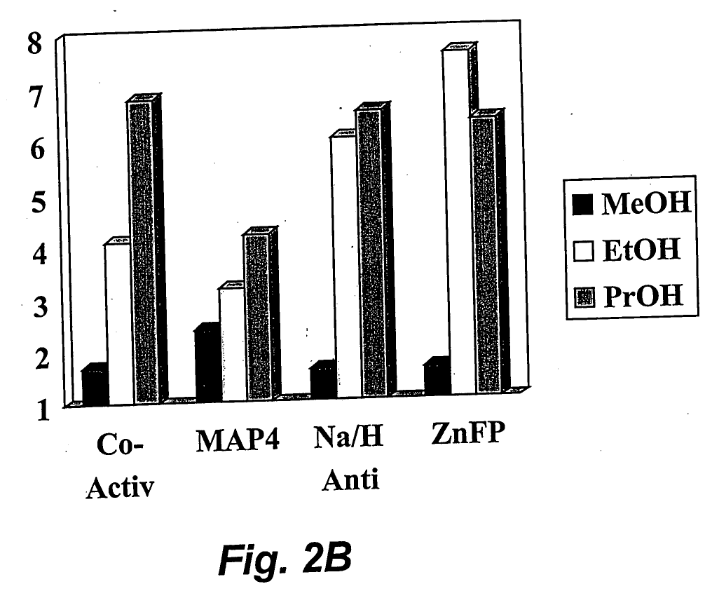

FIG. 2A depicts the results from an initial study on the effects of ethanol on gene expression levels in SHSY5Y cells. Hybridization strength is given a baseline of 1 and increased intensity is expressed in as a multiple of the baseline, i.e., 2-fold, 3-fold, 4-fold etc. The hybridization intensity has been shown to be proportional to expression level. At an ethanol concentration of 50 mM, DBH (dopamine b-hydroxylase) shows a 3 fold greater hybridization intensity than the control, while PDGFR, DLK, GABA-β3, PTK and NPTX2 all hybridize at just over the baseline. At an ethanol concentration of 100 mM, DBH increases to a 5 fold hybridization intensity over the baseline, while DLK, PTK and PDGFR, have increased to 3 fold and GABA-β3, and NPTX2 are around 2 fold. At an ethanol concentration of 150 mM, DBH hybridization intensities have risen to nearly 9 fold the baseline, DLK is at nearly 7 fold, and PDGFR is at 5 fold. Interestingly, PTK levels reduce to 2 fold, while GABA-β3, and NPTX2 remain at 2 fold the baseline level of hybridization.

FIG. 2B depicts the response of different types of cells in response to 50 mM concentrations of methanol, ethanol and propanol in order to demonstrate the pharmacological specificity of the early (2 hour) responses to ethanol. In Co-Activ, exposure to methanol resulted in only a 1.5-fold increase in hybridization, while ethanol resulted in a 4-fold increase and propanol resulted in over a 6.5-fold increase. In MAP4, methanol exposure resulted in over a 2-fold increase, while ethanol resulted in a 3-fold increase and propanol resulted in a 4-fold increase. In Na/H Anti, methanol exposure resulted in a 1.5-fold increase in hybridization while ethanol rose to a nearly 6-fold increase in hybridization and propanol resulted in a 6.5-fold increase. In ZNFP, methanol exposure resulted in a 1.5 fold increase in hybridization while ethanol exposure resulted in a 7.5-fold exposure and propanol resulted in just over a 6-fold hybridization increase.

Table 8 portrays the numbers of genes, listed in Table 5 from the SHSY-5Y human cell line that responded to acute ethanol exposure, and their functional groups. Column 1 lists the presumed functional class of the gene. Column 2 enumerates the number of genes from Table 5 that increased in expression by 1.5-fold or more fold following a 2 hr ethanol (100 mM) exposure. Column 3 enumerates the number of genes from Table 5 that decreased in expression.

TABLE 8
Acute ethanol-responsive genes in SHSY-5Y cells.

| Class | Increases | Decreases |
|---|---|---|
| Cell division | 1 | 0 |
| Cell Signaling | 9 | 9 |
| Cell structure | 0 | 1 |
| Defense/homeostasis | 1 | 3 |
| Gene/protein expression | 7 | 4 |
| Metabolism | 3 | 3 |
| Unclassified | 1 | 3 |
| Totals: | 22 | 23 |

Table 9 portrays the numbers and ways in which the genes listed in Table 5 from the SHSY-5Y human cell line responded to chronic ethanol exposure (72 hr, 100 mM ethanol). Columns are similar to Table 8.

TABLE 9
Chronic ethanol-responsive genes in SHSY-5Y cells.

| Class | Increases | Decreases |
|---|---|---|
| Cell division | 0 | 0 |
| Cell Signaling | 13 | 3 |
| Cell structure | 2 | 1 |
| Defense/homeostasis | 0 | 0 |
| Gene/protein expression | 3 | 3 |
| Metabolism | 3 | 1 |
| Unclassified | 1 | 1 |
| Totals: | 22 | 9 |

### Example 3

### Effect(s) of Ethanol on SHSY-5Y Cells in Culture

Ethanol is one of the most commonly used and abused drugs worldwide. Like opioids, amphetamines or nicotine, upon chronic exposure, ethanol produces behavioral adaptations including tolerance, sensitization, dependence and craving. While in recent years dramatic progress has been made in understanding its acute effects in the central nervous system (CNS), molecular mechanisms underlying the development of alcohol addiction remain poorly understood. In contrast to most drugs of abuse that act by binding to a specific receptor, ethanol appears to affect the function of multiple neurotransmitter systems. Thus, acute ethanol has been shown to inhibit activation of excitatory NMDA receptor, opioid receptors and L-type voltage gated calcium channels and to potentiate activation of inhibitory γ-aminobutyric acid type A (GABA_{A}) receptor and serotonin 5HT3 receptor. Numerous studies have also described an effect of ethanol on signaling cascades such as cyclic AMP (cAMP) and phosphoinositide/calcium pathways.

Acute modifications of neurotransmitter systems and signal transduction pathways by ethanol have been hypothesized to ultimately contribute to the molecular events involved in the development of tolerance to and dependence on alcohol by triggering changes in gene expression. Alterations in the expression of selective subunits of GABA_{A} and NMDA receptors are among the most frequently reported changes associated with chronic ethanol exposure. Increase expression of voltage-dependent calcium channels and 6 opioid receptor and decrease levels of mRNA coding for the α subunit of the stimulatory GTP-binding protein Gs have also been described. While these latter studies have mainly concentrated on genes coding for signaling molecules known to be functionally regulated by ethanol, it is very likely that many other genes are affected by chronic exposure to this drug. Such genes couldn't be identified using a candidate gene approach.

Therefore, in the present study, we sought to characterize the effect of prolonged ethanol treatment on gene expression in neuronal cells using a non-biased approach. We used the recently developed oligonucleotide array technology to monitor simultaneously the expression levels of nearly 6000 genes in response to ethanol in human neuroblastoma SH-SY5Y cells. This cell line represents a useful human preparation that is well suited for mechanistic studies of ethanol-induced changes in gene expression. SH-SY5Y cells have been shown to display many features of mature noradrenergic neurons including the ability to uptake and release norepinephrine (NE) and have previously been used to investigate cellular effects of various drugs of abuse such as opioids, nicotine or ethanol.

Analysis of gene expression profiles in response to ethanol after 3 days treatment led to the identification of 42 genes differentially regulated in SH-SY5Y cells. In particular, we identified 4 genes whose expressions changed in a dose-dependent manner in response to ethanol and we believe represent important targets of ethanol. These genes encoded, respectively, for dopamine β hydroxylase (DBH) the enzyme catalyzing the formation of NE, sodium-dependent NE transporter (NE) involved in re-uptake of this neurotransmitter, delta-like protein (DLK), an EGF-like transmembrane protein and monocyte chemoattractant peptide 1 (MCP-1) a chemokine of the C-C family.

Methods.

Oligonucleotide Arrays.

Gene expression levels were monitored using Affymetrix GeneChip Hu6800 set including 4 probe arrays (A, B, C, D) of over 65000 different oligonucleotides each. Oligonucleotides are complementary to 5800 full-length human cDNA based on sequence information from the UniGene, GenBank and TIGR databases. Each gene is represented by an average of 20 different pairs of 20-25 mer oligonucleotides. Each pair consists of a perfectly complementary oligonucleotide (referred to as perfect match, PM) and a closely related mismatch oligonucleotide (MM) identical to its PM partner except for a single base difference in the central position. The MM probe of each pair serves as an internal control for hybridization specificity.

Cell Culture and Animals.

Cell culture experiments used the human neuroblastoma cell line SH-SY5Y-AH1861 (passage number 7). Cells were routinely grown at 37° C. in DMEM supplemented with 2 mM glutamine and 10% (vol/vol) fetal bovine serum in a humidified atmosphere of 10% CO₂ in air. For gene expression analysis, cells were treated for 72 h in the absence or presence of 50, 100 or 150 mM ethanol. Culture media were renewed every 24 h.

Animal studies were conducted on female DBA/2 mice (Simonsen Laboratories, Gilroy, Calif.) weighing 20-30 g at 8 weeks of age. All animals were housed individually under a 12:12 light-dark cycle at 22° C. and given ad libitum access to food and water before and after injection procedures. Animals were injected intraperitoneally with 4 g/kg ethanol or saline at 10:00 am, returned to their home cage, and killed 6 or 24 h later by cervical dislocation and subsequent decapitation. Adrenal glands were dissected out, immediately frozen into liquid nitrogen and stored at −80° C. until needed. All experiments were performed in accordance with the National Institutes of Health Guide for the Care and Use of Laboratory Animals and institutional guidelines.

cRNA Preparation for Array Hybridization.

Following ethanol treatment, cells were trypsinized and washed in ice-cold Phosphate Buffer Saline (PBS). Poly A⁺-RNA was directly extracted from cell pellets (30 to 40×10⁶ cells) using the Pharmacia Quick mRNA Prep kit or the Qiagen Oligotex direct mRNA kit. Poly A⁺-RNA were then reverse-transcribed into double stranded cDNA using the GIBCO BRL Superscript Choice system. Priming of the first-strand cDNA synthesis was performed with a T7-(dT)₂₄ oligomer containing the promoter of the T7 polymerase (5′-GGC CAG TGA ATT GTA ATA CGA CTC ACT ATA GGG AGG CGG-(dT)₂₄-3′ (GENSET, SEQ ID NO:1). Double stranded cDNA was subsequently purified by phenol/chloroform extraction and ethanol precipitation. Ambion's T7 MEGAscript kit was used to produce biotin-labeled cRNA from cDNA. The reaction was carried out with 0.5 to 1 μg of starting cDNA in the presence of a mixture of unlabeled ATP, CTP, GTP and UTP and biotin-labeled CTP and UTP (biotin-11-CTP and biotin-16-UTP, ENZO Diagnostics). Labeled-cRNA was purified on affinity resin (RNAeasy, Qiagen) and quantified by absorbance at 260 nm. Prior to hybridization, 10 μg of cRNA were fragmented randomly to an average size of 50-100 bases by incubating at 94° C. for 35 min in 40 mM Tris-acetate pH 8.1, 100 mM potassium acetate and 30 mM magnesium acetate.

Array Hybridization and Scanning.

Hybridizations were carried out as described in (Lockhart et al. (1996) Nature Biotechnology, 14: 1675) or the standard hybridization procols provided by Affymetrix with their GeneChip™ kits). Briefly, aliquots of fragmented cRNA (10 μg in a 200 μl master mix) were hybridized to Hu6800 Gene Chip arrays at 40° C. for 16 h in a rotisserie oven set at 60 rpm. Following hybridization, arrays were washed with 6×SSPE and 0.5×SSPE containing 0.005% Triton X-100, and stained with streptavidin-phycoerythrin (Molecular probes). After removal of the excess of dye, arrays were read using a specially designed confocal microscope scanner (Affymetrix, Santa Clara, Calif.).

Data Analysis.

Absolute and comparison analyses were conducted using the GENECHIP Software 3.1. The total intensity of all chips was scaled to a uniform value by normalizing the average intensity of all genes (total intensity/number of genes) to a fixed value of 74. Under these conditions, the scaling factor for all chips varied between 0.5 and 2.

Northern Blot and Reverse Transcriptase-PCR (RT-PCR) Analysis.

Total RNA was isolated from control and ethanol-treated cells and analyzed by formaldehyde-agarose gel electrophoresis and Northern blot hybridization to confirm oligonucleotide array results. RNA blots were probed with ³²P-labeled inserts of human DBH, NET, DLK and MCP-1 cDNAs. Probes were synthesized by RT-PCR using SH-SY5Y total RNA as template. PCR primers were: 5′-CCT CAC TGG CTA CTG CAC GG-3′ (SEQ ID NO:2) and 5′-CTC TTC CAG TGT GGA GAT G-3′ (SEQ ID NO:3) for DBH, 5′-AGA AGA ATC ACC AGC AGC AAG TG-3′ (SEQ ID NO:4) and 5′-GGT GCC TCA GTT TTC CCA TTG-3′ (SEQ ID NO:5) for MCP-1,5′-GCA TTG CGT TTG TCA CAC AGC-3′ (SEQ ID NO:6) and 5′-CTG TGG GTA TCG TCT TCC C-3′ (SEQ ID NO:7) for DLK, and 5′-GGA GCT GGC CTA GTG TTC-3′ (SEQ ID NO:8) and 5′-CCA TAG GCC AGT CTC TCC C-3′ (SEQ ID NO:9) for NET. Human GAPDH cDNA probe (Clonetech) was used as an internal control for total RNA normalization.

Semi-quantitative RT-PCR was used to determine the effect of ethanol on DBH expression in vivo, in adrenal glands of acutely treated mice. Total RNA extracted from saline or ethanol-treated mice were transcribed into single stranded cDNAs (ss cDNAs) using the GIBCO BRL Superscript Choice system. Aliquot of ss cDNA were then used in comparative PCR. Mouse DBH primer pair was 5′-CTT GGA AGA GCC ATT TCA GTC GCT G-3′ (SEQ ID NO:10) and 5′-CAT TTT GGA GTC ACA GGG TCC GTT G-3′ (SEQ ID NO:11). We performed duplex reaction using GAPDH as an endogenous amplification standard. PCR conditions were optimized so that the amplification of both, GAPDH and DBH cDNAs were in the exponential phase. PCR primers for GAPDH were obtained form Clonetech.

Western Blot and Enzyme Linked Immunoabsorbent Assay (ELISA).

The relative amount of DBH protein between whole cell homogenates of control and ethanol-treated cells was determined by Western blot analysis following standard protocols using a polyclonal antibody (Calbiochem). MCP-1 production was monitored in the culture media of cells treated in the absence or presence of ethanol using the Quantikine MCP-1 immunoassay from R&D System.

High Performance Liquid Chromatography (HPLC).

Culture media from cells treated in the absence or presence of ethanol were analyzed for norepinephrine content by reversed-phased HPLC with electrochemical detection according to standard procedures (see Gamache et al. (1993) J. Chromatogr. B Biomed. Appl., 614: 213-220.). All HPLC apparatus was from ESA, Inc. (Chelmsford, Mass.). Following precipitation with 0.1 M perchloric acid and centrifugation over a 5000 MW cut-off centrifugal filter, culture media (10 μl aliquot) was injected onto an ESA HR-80 column (C-18, 4.6 mm×8 cm, 3 um particle size) using a Model 540 refrigerated autosampler injector and a Model 580 solvent delivery pump. Mobile phase consisted of 75 mM sodium acetate trihydrate, 1.5 mM sodium dodecyl sulfate, 100 μl/l triethylamine, 25 μM EDTA, 12.5% acetonitrile, 12.5% methanol, pH 5.6, filtered through a 0.22 μm nylon membrane. Eluents were detected at a flow rate of 1.0 ml/min using a Model 5011 analytical cell with palladium reference electrode, a Model 5020 guard cell, and a Model 5200A Coulochem II electrochemical detector. Electrode settings were +350 mV for the guard cell, −100 mV for the pre-oxidation electrode, and +280 mV for the detection electrode. Samples were analyzed at 5 nA sensitivity and compared with a two-point monoamine standard calibration curve at 1 and 5 pg/μl using the Model 501 analysis software package.

Results

Selection of mRNA Differentially Regulated by Ethanol in SH-SY5Y Cells.

SH-SY5Y cells were treated for 72 h in the absence or presence of 50, 100 or 150 mM ethanol in duplicate experiments (experiments #1 and #2). Gene expression profiles were generated by hybridization to oligonucleotide microarrays as described under methods. Between 2000 and 2500 genes were detected in this cell line under our experimental conditions. To identify genes differentially regulated by ethanol, we compared the relative abundance of mRNA between the control sample and each ethanol-treated sample in a given experiment. In experiment #1, cRNA prepared from untreated and 100 mM ethanol-treated cells were hybridized twice. An additional comparison file was created from these repeat hybridizations and was included in the analysis.

Due to its low potency, we anticipated that ethanol would induce changes in mRNA levels of low amplitude. Indeed, previous studies have mainly reported changes in gene expression under 2 fold in response to ethanol. Consequently, we choose to look for genes those expression levels deviated from that in control cells by >=1.5 or <=−1.5 fold in treated cells. Genes were selected only if they met this criterion in at least 4 out of the 7 comparison files created, 2 per experiment. Under these conditions, we identified 500 genes that were empirically subjected to a more stringent filtering using different parameters from Affymetrix algorithm. Thus, only genes flagged as “increased” or “decreased” at least once in both experiments at any ethanol concentration were further selected. In addition, genes flagged as “increased” had to be called “present” at least once in any ethanol-treated samples in both experiments and genes identified as “decreased” had to be called “present” in one control sample from each experiment. A final selection was done to eliminate transcripts that met all the above criteria but for which the average intensity was derived from hybridization to a low number of probe pairs on the array (<10). Under these conditions, we identified 18 genes down regulated and 24 genes up regulated by ethanol.

FIG. 3A illustrates the response of these 42 genes to 72 h treatment with 100 mM ethanol. Among them, only 1 was previously described as regulated by alcohol in SH-SY5Y cells. This gene, downregulated by ethanol, encoded the α7 subunit of the neuronal acetylcholine receptor (nAChRα7). Genes were ordered on the basis of their known cellular function. A majority of them (26%) encoded signaling molecules such as membrane receptors, ligands or enzymes. A significant group of genes including those coding glutathione-5-transferase (GST) and neuronal inhibitory apoptosis peptide (Niap) was found to be involved in protection against oxidative stress or apoptosis. 85% of the genes affected had a low level of expression with an average intensity below 100 in baseline condition. Among the higher abundant genes were those encoding matrix Gla protein (MGP) and secreted protein acidic cystein-rich (SPARC), 2 proteins previously described as major components of the extracellular matrix of bone (basal average intensity of 860 and 470, respectively). Similarly, genes encoding Ly-GDI, an inhibitor of RhoGTPase and the chemokine MCP-1 were significantly expressed in SH-SY5Y cells (basal average intensity of 207 and 405, respectively).

As expected, ethanol didn't induce dramatic changes in mRNA levels. However, the confidence in the changes observed was strengthened by their reproducibility. Thus, DLK, MCP-1 and cytokeratin 18 gene expressions were consistently changed by ethanol in all 7 pair-wise comparisons generated. Eleven genes including those coding DBH, AChRα7, MGP, neuronal inhibitory apoptosis protein (Niap) and MAP kinase phosphatase-1 were differentially regulated in 6 out of the 7 comparison files. DBH gene exhibited the largest change in expression in response to ethanol. Its mRNA levels changed in a dose-dependent manner in response to alcohol with a 5 to 6 fold increase at 100 mM (FIG. 3B). At least 3 other genes coding for DLK, NET and MCP-1 showed a dose-response to ethanol (FIG. 3B). Based on their expression profile, these 4 genes are more likely to represent biologically important targets of ethanol and were therefore studied further.

Ethanol Effect on DBH, NET, DLK and MCP-1 Expression.

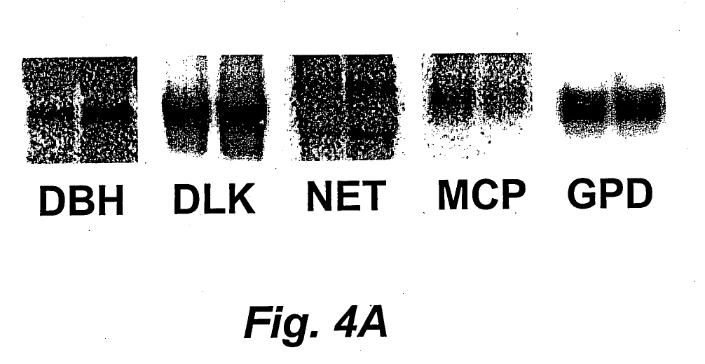

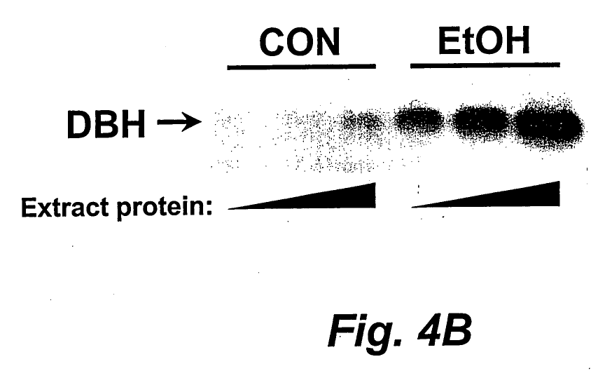

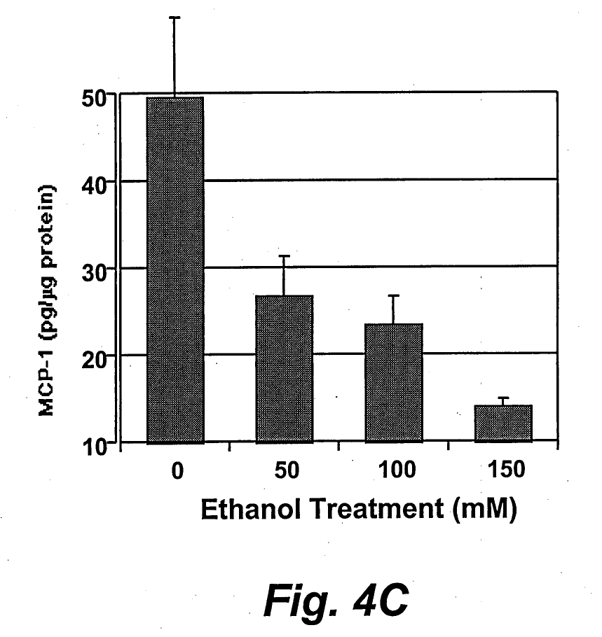

We confirmed ethanol-induced increase in DBH, DLK and NET mRNA levels and decrease in MCP-1 transcript levels after 3 days treatment by Northern blot analysis (FIG. 4A). Changes in expression were detected as early as 24 h after addition of the drug (data not shown). As determined by ELISA, reduction in MCP-1 mRNA levels in the presence of ethanol was accompanied by a decrease in peptide release in the culture media of treated cells (FIG. 4C). Similarly, increased DBH mRNA levels in ethanol-treated cells were correlated with an enhancement in DBH protein expression (FIG. 4B). Increase in DBH protein levels was sustained up to 7 days treatment in the presence of ethanol (data not shown). To determine whether the up-regulation of DBH and NET gene expression in response to ethanol led to altered NE production, we studied the effect of ethanol on endogenous NE release. For this purpose, SH-SY5Y cells were cultured for 3 days in the absence or presence of 150 mM ethanol. Media from the last 24 h incubation were collected and analyzed for their NE content by HPLC. Under these conditions, NE levels were found to be significantly greater in the media of treated cells (23.554+/−5.218 ng/mg of protein, n=3) than in the media of control cells (0.957+/−0.49 ng/mg of protein, n=3). These data suggest that ethanol regulation of DBH expression is biologically relevant.

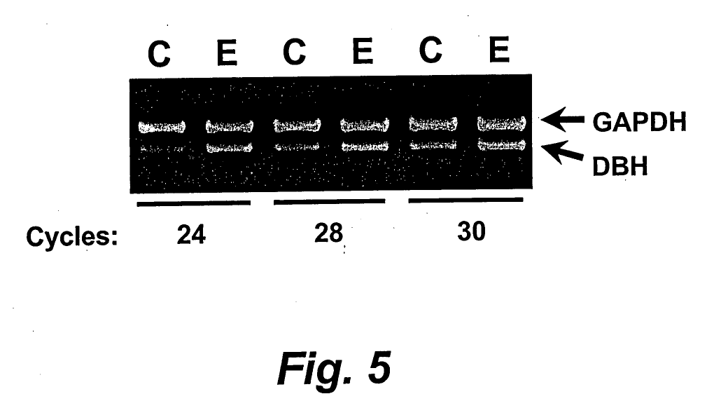

Several lines of evidence indicate that ethanol may modulate noradrenergic function in vivo. Thus, ethanol administration was previously shown to elicit a dose-dependent elevation in plasma norepinephrine levels. In addition, clonidine, an antagonist of noradrenergic receptors was found to strongly reduce ethanol withdrawal syndrome. Finally, several investigators have reported that dopamine beta-hydroxylase inhibitors reduce voluntary ethanol intake in rats. Based on these data and our results, we hypothesized that ethanol may regulate DBH expression in vivo. This enzyme is expressed in restricted regions including the brain locus coeruleus, sympathetic ganglia and adrenal medulla. We investigated the effect of acute ethanol on DBH mRNA levels in both, brain and adrenal glands of DBA/2 mice. DBH transcript levels were monitored 6 or 24 h after injection of a single dose of 4 g/kg ethanol or saline by RT-PCR. A significant increase in DBH mRNA levels was detected in the adrenal of ethanol-treated mice as compared to saline-injected mice 24 h after injection (FIG. 5). No difference in expression was observed at 6 h following injection or at any time point in the brain (data not shown).

Coregulation of DBH, NET, DLK, and MCP-1 by Dibutyryl-Cyclic AMP (db-cAMP).

Since all 4 candidate genes showed a similar response to ethanol over time, we hypothesized that their regulation by ethanol may occur through a common pathway in SH-SY5Y cells. Previous studies have shown that neuroblastoma cells derived from the neural crest could differentiate either towards a more neuronal phenotype in the presence of retinoic acid, or a chromaffin-like phenotype in the presence of glucocorticoids or dibutyryl-cyclic AMP (db-cAMP). Several genes listed in Table 1 were found to be differently regulated during these two differentiation processes. In particular, DLK expression appeared to be specifically induced during chromaffin differentiation. Therefore, we tested the effect of dexamethasone and db-cAMP on the expression of our 4 candidate genes. In contrast to ethanol, after 3 days treatment, 100 nM dexamethasone had a minimal effect on DBH, NET and DLK mRNA levels while it stimulated MCP-1 gene expression (data not shown). On the other hand, treatment in the presence of 1 mM db-cAMP for 3 days produced similar changes in expression to those induced by ethanol (FIG. 5). Furthermore, as observed with ethanol, db-cAMP effects were significant as early as 24 h after treatment (data not shown). Considering previous data showing a stimulatory effect of ethanol on cAMP metabolism in neuronal cells and the similitude of ethanol and db-cAMP actions on DBH, NET, DLK and MCP-1 mRNA levels in SH-SY5Y cells, we propose that ethanol may regulate the expression of these genes through an increase in intracellular cAMP levels.

It is understood that the examples and embodiments described herein are for illustrative purposes only and that various modifications or changes in light thereof will be suggested to persons skilled in the art and are to be included within the purview of this application and scope of the appended claims. All publications, patents, and patent applications cited herein are hereby incorporated by reference in their entirety for all purposes.

What is claimed is:

2. A method of monitoring the response of a cell to a drug of abuse said method comprising:
contacting said cell with said drug of abuse;
providing a biological sample comprising said cell; and
detecting, in said sample, the expression of one or more genes or ESTs selected from the group consisting of the genes and ESTs of Table 1, the genes and ESTs of Table 2, the genes and ESTs of Table 3 the genes and ESTs of Table 4, the genes and ESTs of Table 5, and the genes and ESTs of Table 6, wherein a difference between the expression of one or more of said genes or ESTs in said sample and one or more of said genes or ESTs in a biological sample not contacted with said drug of abuse indicates a response of said cell to the drug of abuse.

3. The method of claim 2, wherein said genes or ESTs are selected from the group consisting of dopamine β-hydroxylase (DBH), sodium-dependent norepinephrine transporter (NET), delta-like protein (DLK), and monocyte chemoattractant peptide 1 (MCP-1).

4. The method of claim 2, wherein said contacting comprises contacting said cell with an alcohol.

5. The method of claim 4, wherein said alcohol is ethyl alcohol.

6. The method of claim 4, wherein said genes or ESTs are selected from the group consisting of the genes and ESTs listed in Table 1.

7. The method of claim 2, wherein said drug of abuse is selected from the group consisting of alcohol, a stimulant, and an opiate.

8. The method of claim 7, wherein said drug of abuse is ethanol or cocaine.

9. The method of claim 7, wherein said drug of abuse is selected from the group consisting of cocaine, amphetamine, methamphetamine, ephenedrine, methylphenidate, and methcathinone.

10. The method of claim 7, wherein said genes or ESTs are selected from the genes or ESTs of Table 6.

11. The method of claim 2, wherein said contacting comprises contacting a cell in culture.

12. The method of claim 2, wherein said contacting comprises contacting a tissue in culture.

13. The method of claim 2, wherein said contacting comprises administering said alcohol or stimulant to an organism.

14. The method of claim 2, wherein said organism is selected from the group consisting of a human, a non-human primate, a rodent, a porcine, a lagomorph, a canine, a feline, and a bovine.

15. The method of claim 2, wherein said biological sample is a tissue sample.

16. The method of claim 2, wherein said detecting comprises detecting a protein fully or partially, encoded by one of said genes or ESTs.

17. The method of claim 16, wherein said detecting is via a method selected from the group consisting of capillary electrophoresis, a Western blot, mass spectroscopy, immunochromatography, and immunohistochemistry.

18. The method of claim 2, wherein said detecting comprises obtaining a nucleic acid from said cell and hybridizing said nucleic acid to one or more probes that specifically hybridize to said genes or ESTs under stringent conditions.

19. The method of claim 18, wherein said hybridizing is according to a method selected from the group consisting of a Northern blot, a Southern blot, an array hybridization, an affinity chromatography, and an in situ hybridization.

20. The method of claim 18, wherein said one or more probes is a plurality of probes that forms an array of probes.

21. The method of claim 20, wherein said array of probes comprises at least 1000 different probes.

22. The method of claim 21, wherein said array comprises at least about 1000 different probes per cm².

23. The method of claim 21, wherein said probes are chemically synthesized oligonucleotides covalently linked to a solid support.

24. The method of claim 21, wherein said probes are spotted onto a solid support.

25. The method of claim 21, wherein said array of probes additionally includes one or more probes that specifically hybridize to a housekeeping gene.

26. The method of claim 25, wherein said housekeeping gene is selected from the group consisting of an actin gene, and a G6PDH gene.

27. A method of screening for an agent that alters the response of a cell to a drug of abuse, said method comprising:
contacting said cell with said drug of abuse;
contacting said cell with said agent;
providing a biological sample comprising said cell;
detecting, in said sample, the expression of one or more genes or ESTs, selected from the group consisting of the genes and ESTs of Table 1, the genes and ESTs of Table 2, the genes and ESTs of Table 3 the genes and ESTs of Table 4 the genes and ESTs of Table 5, and the genes and ESTs of Table 6, wherein a difference in the expression level of one or more of said genes or ESTs in said sample, as compared to said genes or ESTs in a sample not contacted with said test agent indicates that the test agent alters the response of said cell to the drug of abuse.

28. The method of claim 27, wherein said genes or ESTs are selected from the group consisting of dopamine β-hydroxylase (DBH), sodium-dependent norepinephrine transporter (NET), delta-like protein (DLK), and monocyte chemoattractant peptide 1 MCP-1).

29. The method of claim 27, wherein said contacting comprises contacting said cell with an alcohol.

30. The method of claim 29, wherein said alcohol is ethyl alcohol.

31. The method of claim 29, wherein said genes or ESTs are selected from the group consisting of the genes and ESTs of listed in Table 1.

32. The method of claim 27, wherein said drug of abuse is selected from the group consisting of alcohol, a stimulant, and an opiate.

33. The method of claim 32, wherein said drug of abuse is ethanol or cocaine.

34. The method of claim 32, wherein said drug of abuse is selected from the group consisting of cocaine, amphetamine, methamphetamine, ephenedrine, methylphenidate, and methcathinone.

35. The method of claim 32, wherein said genes or ESTs are selected from the genes or ESTs of Table 6.

36. The method of claim 27, wherein said contacting comprises contacting a cell in culture.

37. The method of claim 27, wherein said contacting comprises contacting a tissue in culture.

38. The method of claim 27, wherein said contacting comprises administering said alcohol or stimulant to an organism.

39. The method of claim 27, wherein said organism is selected from the group consisting of a human, a non-human primate, a rodent, a porcine, a lagomorph, a canine, a feline, and a bovine.

40. The method of claim 27, wherein said biological sample is a tissue sample.

41. The method of claim 27, wherein said detecting comprises detecting a protein fully or partially, encoded by one of said genes or ESTs.

42. The method of claim 41, wherein said detecting is via a method selected from the group consisting of capillary electrophoresis, a Western blot, mass spectroscopy, immunochromatography, and immunohistochemistry.

43. The method of claim 27, wherein said detecting comprises obtaining a nucleic acid from said cell and hybridizing said nucleic acid to one or more probes that specifically hybridize to said genes or ESTs under stringent conditions.

44. The method of claim 43, wherein said hybridizing is according to a method selected from the group consisting of a Northern blot, a Southern blot, and array hybridization, an affinity chromatography, and an in situ hybridization.

45. The method of claim 43, wherein said one or more probes is a plurality of probes that forms an array of probes.

46. The method of claim 45, wherein said array of probes comprises at least about 1000 different probes.

47. The method of claim 46, wherein said array comprises at least about 1,000 different probes per cm².

48. The method of claim 46, wherein said probes are chemically synthesized oligonucleotides covalently linked to a solid support.

49. The method of claim 46, wherein said probes are spotted onto a solid support.

50. The method of claim 46, wherein said array of probes additionally includes one or more probes that specifically hybridize to a housekeeping gene.

51. The method of claim 50, wherein said housekeeping gene is selected from the group consisting of an actin gene, and a G6PDH gene.

52. A nucleic acid array for monitoring the response of a cell to alcohol or to a stimulant said array comprising a plurality of nucleic acid probes attached to a solid support, said array predominantly containing nucleic acid probes that hybridize under stringent conditions to nucleic acids selected from the group consisting of the genes and ESTs of Table 1, the genes and ESTs of Table 2, the genes and ESTs of Table 3 the genes and ESTs of Table 4 the genes and ESTs of Table 5, and the genes and ESTs of Table 6.

53. The array of claim 52, wherein said array comprises probes that hybridize under stringent conditions to a nucleic acid selected from the group consisting of dopamine β-hydroxylase (DBH), sodium-dependent norepinephrine transporter (NET), delta-like protein (DLK), and monocyte chemoattractant peptide 1 (MCP-1).

54. The array of claim 52, wherein said array of probes comprises at least about 1,000 different probes.

55. The array of claim 54, wherein said array comprises at least about 1,000 different probes per cm².

56. The array of claim 54, wherein said probes are chemically synthesized oligonucleotides covalently linked to a solid support.

57. The array of claim 54, wherein said probes are spotted onto a solid support.

58. The array of claim 54, wherein said array of probes additionally includes one or more probes that specifically hybridize to a housekeeping gene.

59. The array of claim 54, wherein said array of probes additionally includes a mismatch control probe.

60. The array of claim 58, wherein said housekeeping gene is selected from the group consisting of an actin gene, and a G6PDH gene.

61. A method of making a nucleic acid probe array for monitoring the response of a cell to alcohol or to a stimulant said method comprising:
attaching to a surface, one or more nucleic acid probes that specifically hybridize to a nucleic acid selected from the group consisting of the genes and ESTs of Table 1, the genes and ESTs of Table 2, the genes and ESTs of Table 3 the genes and ESTs of Table 4 the genes and ESTs of Table 5, and the genes and ESTs of Table 6.

62. The method of claim 61, wherein said array comprises probes that hybridize under stringent conditions to a nucleic acid selected from the group consisting of dopamine β-hydroxylase (DBH), sodium-dependent norepinephrine transporter (NET), delta-like protein (DLK), and monocyte chemoattractant peptide 1 (MCP-1).

63. The method of claim 61, wherein said probes are chemically synthesized oligonucleotides covalently linked to a solid support.

64. The method of claim 61, wherein said probes are spotted onto a solid support.

65. The method of claim 61, wherein said array of probes comprises at least about 1,000 different probes.

66. The method of claim 61, wherein said array comprises at least about 1,000 different probes per cm².

67. The method of claim 61, wherein said array of probes additionally includes one or more probes that specifically hybridize to a housekeeping gene.

68. The array of claim 67, wherein said housekeeping gene is selected from the group consisting of an actin gene, and a G6PDH gene.

69. The method of claim 61, wherein said array of probes additionally includes a mismatch control probe.

70. A nucleic acid construct comprising
a nucleic acid probe selected from the group consisting of the genes and ESTs of Table 1, the genes and ESTs of Table 2, the genes and ESTs of Table 3 the genes and ESTs of Table 4 the genes and ESTs of Table 5, and the genes and ESTs of Table 6;
an origin or replication; and
a promoter.

71. A vector comprising the construct of claim 70.

72. A composition comprising the vector of claim 71 and a carrier.

73. A host cell transfected with the nucleic acid construct of claim 70.

74. A host cell transfected with the vector of claim 71.

75. A method of amplifying a probe, said method comprising:
culturing the host cell of claim 73 in a growth medium and under amplifying conditions; and
allowing the construct to accumulate.

76. The method of claim 75, further comprising separating the construct from the medium and the cells.


SEQUENCE LISTING (11 sequences)

SEQ ID NO: 1 - DNA, 39 bp, Artificial
  Synthetic oligonucleotide primer.
ggccagtgaattgtaatacgactcactatagggaggcgg

SEQ ID NO: 2 - DNA, 20 bp, Artificial
  Synthetic oligonucleotide primer.
cctcactggctactgcacgg

SEQ ID NO: 3 - DNA, 19 bp, Artificial
  Synthetic oligonucleotide primer.
ctcttccagtgtggagatg

SEQ ID NO: 4 - DNA, 23 bp, Artificial
  Synthetic oligonucleotide primer.
agaagaatcaccagcagcaagtg

SEQ ID NO: 5 - DNA, 21 bp, Artificial
  Synthetic oligonucleotide primer.
ggtgcctcagttttcccattg

SEQ ID NO: 6 - DNA, 21 bp, Artificial
  Synthetic oligonucleotide primer.
gcattgcgtttgtcacacagc

SEQ ID NO: 7 - DNA, 19 bp, Artificial
  Synthetic oligonucleotide primer.
ctgtgggtatcgtcttccc

SEQ ID NO: 8 - DNA, 18 bp, Artificial
  Synthetic oligonucleotide primer.
ggagctggcctagtgttc

SEQ ID NO: 9 - DNA, 19 bp, Artificial
  Synthetic oligonucleotide primer.
ccataggccagtctctccc

SEQ ID NO: 10 - DNA, 25 bp, Artificial
  Synthetic oligonucleotide primer.
cttggaagagccatttcagtcgctg

SEQ ID NO: 11 - DNA, 25 bp, Artificial
  Synthetic oligonucleotide primer.
cattttggagtcacagggtccgttg